(19) United States
(12) Patent Application Publication
(10) Pub. No.: US 2013/0052654 A1
(43) Pub. Date: Feb. 28, 2013

(54) Amyloid Precursor Protein E2 Domain and Uses Thereof

(71) Applicants: Sven Dahms, Jena (DE); Manuel Than, Jena (DE); Dirk Roeser, Jena (DE); Ina Koennig, Jena (DE)

(72) Inventors: Sven Dahms, Jena (DE); Manuel Than, Jena (DE); Dirk Roeser, Jena (DE); Ina Koennig, Jena (DE)

(73) Assignee: Leibniz Institute For Age Research - Fritz Lipmann Institute (FLI), Jena (DE)

(21) Appl. No.: 13/599,187

(22) Filed: Aug. 30, 2012

Related U.S. Application Data
(60) Provisional application No. 61/528,946, filed on Aug. 30, 2011.

(51) Int. Cl. C12Q 1/37; C12Q 1/56; G01N 33/53; G06G 7/58; C07K 7/08; C07K 14/00; C07K 16/18; C07H 21/02; G01N 33/50; C07K 7/06

(52) U.S. Cl. 435/7.1; 436/86; 435/23; 435/13; 435/24; 530/329; 530/328; 530/327; 530/326; 530/325; 530/324; 530/350; 530/389.1; 536/23.1; 536/24.5; 703/12

(57) ABSTRACT

Members of the Amyloid Precursor Protein family (APP molecules) and their neurotoxic cleavage product Aβ are key players in the development of Alzheimer's disease (AD). Proteolytic processing of APP molecules is influenced by metal ions, protein ligands and its oligomerization state. X-ray structures of the metal bound molecule at 2.6-2.0 Å resolution are presented, providing structural and functional bases for the regulation of APP molecules using conformational information. A metal-dependent molecular switch located within the E2 domain of APP coinciding with a high affinity copper and zinc binding site within the monomeric E2 domain was evaluated. The metal specific coordination spheres of this E2 domain comprise four evolutionary conserved histidine residues. Metal binding induces large conformational changes relative to the metal free protein. This conformational change provides a basis for influencing APP molecule processing and thus trafficking and the production of Aβ.

### CROSS-REFERENCE TO RELATED APPLICATIONS

The present invention claims priority to U.S. provisional application U.S. 61/528,946, filed Aug. 30, 2011, which is incorporated herein by reference in its entirety.

### INCORPORATION OF SEQUENCE LISTING

The instant application contains a Sequence Listing, which has been submitted via EFS-Web and is hereby incorporated by reference in its entirety. Said ASCII copy, created on Aug. 29, 2012 is named 3035-117NP-Sequence-Listing.txt and is 72 kilobytes in size.-

### FIELD OF THE INVENTION

The present invention relates to a method for selecting agents that induce a conformational shift in isolated amyloid precursor protein (APP) molecules as well as a computer and computer implemented methods using structural coordinates of the M1 binding site of the APP-E2 core domain to evaluate the ability of a chemical entity to bind to the APP-E2 core domain.

### BACKGROUND OF THE INVENTION

Alzheimer's disease (AD) is worldwide the most common form of dementia counting more 26 million patients (Barker et al., 2002; Wimo et al., 2010). AD is associated with the deposition of senile plaques and neurofibrillary tangles within the brain. The senile plaques result from the aggregation of neurotoxic Aβ peptide species, the product of sequential proteolysis of the amyloid precursor protein by β- and γ-secretases. Evidence suggests that AD is the result of an imbalanced processing of APP molecules (Blennow et al., 2006; Haass and Selkoe, 2007; Lichtenthaler et al., 2011). It has been observed, both in AD-patients and in APP-transgenic mice, that the homeostasis of the divalent transition metals copper and zinc is imbalanced. Manipulations of intracellular copper has been shown to influence the regulated intramembrane proteolysis (RIP) of APP as well as the production of Aβ. This suggested a role of APP in metal metabolism and led to, among others, metal chelation therapy as possible anti-AD treatment (Bayer and Multhaup, 2006; Duce and Bush, 2010; Hung et al., 2010; Watt et al., 2010).

The publications and other materials, including patents and sequence accession numbers such as UNIPROT and GENEBANK accession numbers, used herein to illustrate the invention and, in particular, to provide additional details respecting the practice are incorporated herein by reference. For convenience, the non-patent publications are referenced in the following text by author and date and are listed alphabetically by author in the appended bibliography.

The members of the APP family of proteins, generally referred to herein as APP molecules, comprise a number of distinct, largely independent structural domains. The extracellular region, which is much larger than the intracellular region, is divided into the E1 and E2 domains, linked by a potentially flexible, less conserved linker region (“acidic linker”) of unknown function, which is primarily composed of acidic amino acids (AcD). Both E1 and the E2 domains have a high degree of sequence conservation. Two tightly interacting subunits constitute the E1 domain (Dahms et al., 2010), also called growth factor like domain (GFLD) or copper binding domain (CuBD). A second linker of undefined structure, containing the cleavage sites of α- and β3-secretases, anchors the whole extracellular part to a single transmembrane helix. Some APP molecules including APLP2 isoforms feature an additional Kunitz-type protease inhibitor domain (KPI) located between the acidic linker and the E2 domain (FIG. 8, see (Reinhard et al., 2005)).

The E2 domain is characterized by a fold that is highly flexible, resulting in large conformational differences of the available E2 structures (Hoopes et al., 2010; Lee et al., 2011; Wang and Ha, 2004). As a result, partially disordered crystals (Keil et al., 2004), anisotropic crystals and high overall B factors of the structures (Hoopes et al., 2010; Lee et al., 2011; Wang and Ha, 2004) have complicated X-ray crystallography studies. A NMR-study also showed that only a part of the E2 domain is rigidly folded in solution. The full length domain is readily degraded by proteases, suspected to allow the protein to adapt to different, yet unknown, functional states (Dulubova et al., 2004).

The question whether metal ion mediated effects in APP physiology (Cappai et al., 2005; Duce et al., 2010; Multhaup et al., 1994) relate to metal homeostasis defects in AD-pathology has remained elusive. Initially, binding sites for copper and zinc ions were identified close to the C-terminus of the APP-E1 domain by peptide mapping (Bush et al., 1993; Hesse et al., 1994). Later His147, His151 and Tyr168 were described as copper coordination sphere of the CuBD (Kong et al., 2007), see also US Patent Publication 2007/0015688. However, the Cu²⁺ binding site is partially obstructed by the tightly associated GFLD (Dahms et al., 2010). Binding of copper to crystals of the isolated CuBD was only observed at high concentrations and in one of the two investigated crystal forms (Kong et al., 2007). Also, the zinc binding peptide, mapped at amino acids 181-200, showed weaker binding than full length APP (Bush et al., 1993).

Thus, there is a need in the art to identify further and more relevant metal binding sites within the APP family of proteins. There is also a need to understand the conformational changes associated with metal binding at those binding sites and to influence or mimic those conformational changes to alter the cleavage pattern of APP molecules.

To address these and other needs apparent to the person skilled in the art, the structure and biochemical properties of the APP-E2 core domain as well as its changes in conformation in presence and absence of metals was analyzed. A crystal structure of the APP-E2 domain in the presence of different metal ions could be defined. In particular, a metal binding site within the structure of the APP-E2 core domain was identified as well as a metal-dependent conformational switch regulating the conformation, flexibility and hence the physiological function of APP molecules.

### SUMMARY OF THE INVENTION

The present invention is in one aspect directed at a method for selecting an agent that influences the conformation of an isolated amyloid precursor protein (APP) E2 core domain comprising

providing a sample comprising said APP-E2 core domain having a first conformation, adding a candidate agent to said sample to induce either an alteration or no alteration in said first conformation,
determining whether said candidate agent induces said alteration in said first conformation to result in a second conformation of said APP-E2 core domain, and
selecting said agent, wherein said agent is the candidate agent that induces said alteration of said first conformation to result in said second conformation.

The first conformation may be an unrestricted conformation and said second conformation may be a restricted conformation. The first conformation may also be a restricted conformation and said second conformation may be an unrestricted conformation. The first conformation may be a first restricted conformation and said second conformation may be a second restricted conformation and vice versa. The APP-E2 core domain may be part of a APP-E2 domain or a full length APP molecule or a fragment thereof comprising the APP-E2 core domain. The alpha helical content of said APP-E2 core domain in said first conformation may be at least 5%, 10%, 20%, 30% or 40% higher or lower than the alpha helical content of said APP-E2 core domain in said second conformation. Whether said candidate agent induces said alteration in said first conformation may be determined by measuring the melting temperature of said APP-E2 core domain subsequent to addition of said candidate agent and comparing said melting temperature to the melting temperature of the APP-E2 core domain prior to addition of said candidate agent or melting temperatures provided for another APP-E2 core domain. Agents may be selected that display melting temperature difference between the first and second conformation of more than 1, 2, 3, 4, 5, 6, 7, 8, 9, 10° C. Whether said candidate agent induces said alteration in said first conformation may also be determined by subjecting said APP-E2 core domain subsequent to addition of said candidate agent to a spectroscopic method to obtain a spectrogram or nuclear magnetic resonance (NMR) to obtain a NMR image and comparing the spectrogram or NMR image with the spectrogram or NMR image of the respective APP-E2 core domain prior to addition of said candidate agent or an NMR image provided for another APP-E2 core domain. In addition, whether said candidate agent induces said alteration in said first conformation may be determined by digesting said APP-E2 core domain subsequent to addition of said candidate agent with a protease and comparing the digestion pattern with a digesting pattern of the respective APP-E2 core domain prior to addition of said candidate agent or digestion patterns provided for another APP-E2 core domain. The APP-E2 core domain may comprise an alpha B helix and an alpha C helix and wherein, in said restricted conformation, an N-terminal part of the alpha C helix and the alpha B helix are rotated by or by more than 5°, 6°, 7°, 8°, 9°, 10°, 11° or 12° around a hinge region in the alpha C helix towards an N-terminal end of the alpha D helix.

The rotation may occur around a hinge region in the alpha C helix. The hinge region may be located C terminally of a histidine residue in the alpha C helix that is part of the metal binding site M1. In said second conformation the alpha B helix and at said alpha C helix may be shifted out of alignment to each other by at least 5° or may remain parallel to each other. A transition from said first conformation to said second conformation or from a second to a first conformation may correlate to a more than 10%, 20%, 30%, 40% or 50% change of protease cleavage at, e.g., an arginine in the alpha C helix of the APP-E2 domain. The sample that comprises said APP-E2 core domain in a unrestricted conformation may be metal free. Prior to adding the candidate agent to said sample a transition metal ion may be added to said APP-E2 core protein to provide said restricted conformation. Said sample may be an in vitro sample comprising said APP-E2 core domain attached to a sensor chip or said APP-E2 core domain in solution. Said APP-E2 core domain may comprise amino acids corresponding to amino acids 301, 303, 312, 313, 334, 338, 349, 360, 361, 367, 368, 382, 385, 386, 388, 391, 395, 397, 404, 405, 410, 419, 424, 425, 428, 432, 439, 448, 459, 462, 467, 473, 477 amino acids of the human APP₆₉₅ protein. The agent may interact with histidine residues corresponding to histidine residues 382, 432 and 436 of human APP₆₉₅. The agent selected may be added to an APP molecule, in particular an isolated and/or purified APP molecule and said APP molecule may be subjected to a protease. The product of this protease treatment (digestion product) may be evaluated. The digestion product may, for example, be the product of a limited proteolysis by trypsin, elastase, thrombin, thermolysin, v8-endoproteinase, aminopeptidase or carboxypeptidase. The digestion product may also be obtained via an in vitro alpha secretase and/or beta secretase assay. The presence of one or more Abeta peptide species may be detected by one or more antibodies that detect said one or more Abeta peptide species.

The invention is in certain embodiments also directed at an agent selected via any one of the above methods. The agent may bind histidine residues corresponding to histidine residues 382, 432 and 436 of the human APP₆₉₅.

The invention is also directed at a method for designing, selecting and/or optimizing a chemical entity that binds to all or part of an APP-E2 core domain as described herein and above comprising:

(a) employing the structural coordinates of the APP-E2 core domain according to any one of FIGS. 1-3 and/or 6 or described elsewhere herein to generate a three-dimensional model of said APP-E2 core domain comprising metal binding site M1 on a computer, wherein said computer comprises the tools for generating said three-dimensional model;
(b) identifying in said model at least three histidines forming a metal binding site M1, wherein the histidines are located in an alpha B, alpha C and alpha D helix of the APP-E2 core domain,
(c) employing the structural coordinates in (a) for designing, selecting and/or optimizing, said chemical entity by performing a conformation shifting operation between said chemical entity which shifts a first conformation of said APP-E2 core domain to a second conformation, wherein in said second conformation:
(i) a distance of at least two of the three histidines widens or narrows by least 2 Å,
(ii) a distance of the alpha C and alpha D helix widens or narrows by least 2 Å, and/or
(iii) an N terminal part of the alpha C helix and the alpha B helix are rotated by or by more than 5°, 6°, 7°, 8°, 9°, 10°, 11° or 12° around a hinge region in the alpha C helix towards an N-terminal end of the alpha D helix. The chemical entity may be a candidate agent or an agent.

In (iii), the alpha C helix and the alpha B helix may have parallel alignment or may be shifted out of alignment during said rotation by at least 5°.

### BRIEF DESCRIPTION OF THE FIGURES

FIG. 1 Cartoon representation of the APP-E2 domain. Intramolecular and intermolecular bound cadmium (CD) ions are shown as dark and light grey spheres, respectively. The different alpha helices progressing on the protein chain from the N- to the C-terminus are shown in different shades. All amino acids involved in metal coordination are shown as sticks. Crystallographic symmetry mates of Cd²⁺-ions were included in the picture to visualize the crystal contacts mediated by cadmium. The rectangles highlight the two intramolecular metal binding sites (M1 and M2), each characterized by a complex coordination sphere.

FIG. 2 Detailed view of the intramolecular metal binding sites M1 and M2. The electron density of the composite annealed 2fo-fc omit map contoured at 0.9 σ is shown as background low densitiy net like structures. (A) cadmium occupied state: Both binding sites (M1 and M2) show multiple conformations of coordinating residues and cadmium ions. Cd²⁺ ions are represented by dark gray spheres. (B) In ZnCl₂ soaked crystals, cadmium is replaced completely by zinc at site M1 and partially at site M2, indicated by the zinc-specific anomalous double difference density map contoured at 7 σ (left net like sphere labeled “Zn”) and the anomalous difference map at low energy representing bound cadmium contoured at 5 σ (on the right larger lighter shaded net like sphere appearing behind the smaller Zn net like sphere; the combination is labeled “Cd/Zn”). (C) In CuCl₂ soaked crystals, cadmium is completely replaced by copper at site M1 but not at site M2, indicated by the copper-specific anomalous double difference density contoured at 7 σ (net like sphere on the left) and the anomalous difference map at low energy contoured at 5 σ (net like sphere on the right).

FIG. 3 Conservation analysis of the metal binding sites. Sequences of vertebrate and invertebrate APP homologues and human APP like proteins (APP molecules) were aligned on the basis of structural similarities between human APP-E2 and C. elegans APL-1. Sequence similarities are highlighted by a low density dotted background, identical amino acids are highlighted by a high density dotted background. The ideogram on top of the alignment indicates α-helical regions in the structure Amino-acids involved in the binding of cadmium ions are designated by numbers corresponding to the respective Cd²⁺-binding site below the alignment. The highly conserved histidines involved in binding of copper and zinc at site M1 are labeled with the numeral “1”.

FIG. 4 Binding of zinc and copper. (A) Metal retention assay probing the capability of the protein to retain metal ions upon concentration. The factor representing the increase in the metal concentration of the retenate (zero corresponds to no retention) is shown in presence of the E2 domain, of lysozyme (control) and in absence of any protein. (B) ITC binding curve of copper to APP-E2. Peak integrals are given as filled squares; curves fitted to a model representing a single set of identical sites are plotted as broken lines. (C) ITC binding curve of zinc to APP-E2. Peak integrals are given as filled squares; curves fitted to a model representing two sets of independent sites are plotted as broken lines. Filled triangles represent integrals for a zinc titration experiment with copper pre-saturated APP-E2. The exothermic binding reaction observed for binding of zinc to APP-E2 is completely suppressed if copper is bound to the protein.

FIG. 5 Conformational differences between metal bound and metal free APP-E2 structures. (A) Superposition of the C-terminal parts of different APP-E2 structures. The human copper-bound (this work), the human metal-free (Wang and Ha, 2004) and the metal-free APL-1 E2 C. elegans homolog structures (Hoopes et al., 2010) are shown in cartoon representation in light grey, dark grey and white, respectively. To mark the metal binding center in the structure, the histidine side chains of the copper coordination sphere are shown as sticks and the central copper ion is indicated by “Cu.” (B) A hinge region in helix α-C causes a concerted, rigid body rotation of helix α-B and the N-terminal half of helix α-C by 12° in the APL-1 E2 domain and the metal bound APP-E2 domain. (C) Fixation of helix α-B in the copper and cadmium bound state of the APP-E2 domain induces a twist of 7° in the N-terminal coiled coil. (D) Detailed view of the copper and zinc binding site M1 of the APP-E2 domain and the structural rearrangements following metal binding. To adopt the position suited for binding of copper, His313 moves towards the coordination center by more than 8 Å in comparison to the metal free structure. In APL-1, His436 is displaced by a bulky arginine residue (Arg380), indicated as R380.

FIG. 6 Stabilization of metal specific conformations of the E2 domain in solution. (A) Limited proteolysis experiments demonstrate the flexibility of the N-terminal part (amino acids 295-385 of APP₆₉₅) in solution, as it is rapidly degraded by Trypsin. Protease cleavage products are marked by arrowheads and the exact cleavage sites determined by Edman sequencing are listed in Table IV. Note the double band ¾ as also evidenced by the sequencing data. Addition of copper to the protein leads to an increased stability of the N-terminal segment (fragment 1), whereas zinc ions lead to the accumulation of the cleavage product 3 and completely abrogate cleavage at Arg385 (corresponding to fragment 4). (B) The N-terminus of the E2 domain is shown in detail according to the B-factors of the cadmium bound structure. The sidechains shown in sphere representation (dark gray) mark the Trypsin cleavage sites in the structure. (C, D, E) Close-up of the region encompassing metal binding site M1 in the metal free form (C) in the copper (D) and in the zinc (E) bound form. The copper coordination sphere in (D) connects helix α-B, α-C and helix α-D, whereas the zinc coordination sphere in (E) connects only helices α-C and helix α-D. (F) The APP-E2 domain shows a typical alpha helical CD-spectrum with a characteristic minimum at 222 nm (insert). Addition of metals (broken lines) to APP-E2 (respective controls represented by solid lines) results in tighter folding of APP-E2 at high temperatures, indicated by CD-melting curves measured at 222 nm. After addition of copper or zinc (dotted lines as indicated in legend) the inflection point representing 50% thermal denaturation is shifted to higher temperatures. Copper addition also results in increased unfolding cooperativity indicated by the increased slope of the melting curve.

FIG. 7 Competitive regulation mechanism proposed for the E2 domain. The complex metal concentration gradients determine the functional state of APP based on their binding to the E2 domain. Dysregulation of the metal homeostasis, e.g. observed in Alzheimer's disease, will affect the equilibrium between zinc and copper and hence the function of the E2 domain.

FIG. 8 Domain substructure of APP isoforms and APP like proteins. Common domains of all APP family members (from N- to C-terminus): E1 domain, acidic domain (AcD), E2 domain, linker, transmembrane region (TM) and APP intracellular domain (AICD). The A beta region, which is released after cleavage of APP by beta- and gamma-secretase, is unique for APP. The Kunitz-type protease inhibitor domain (KPI) is inserted in between the AcD and the E2 domain in APLP2, APP₇₅₁ and APP₇₇₀.

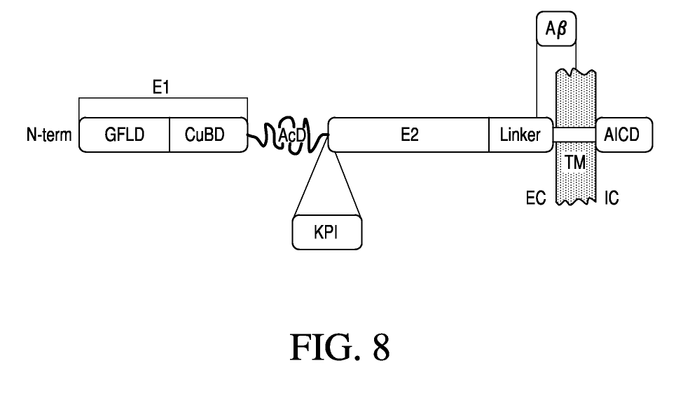

FIG. 9 Oligomerization analysis by GPC coupled SLS. GPC-chromatogram showing the refractive index (RI) and right angle light scattering (RALS) signals for E2 in the absence and the presence of heparin. The data were normalized with respect to the maximum UV absorbance (UV280) to correct for slight differences in concentration. The additional minor features in both RI-traces at higher retention volume result from plain buffer effects. They did not influence the determination of absolute molecular weight and are absent in the respective UV-traces, that were not used for MW-determination due to lower accuracy.

FIG. 10 Additional intermolecular cadmium binding sites become occupied by copper and zinc upon soaking. The anomalous electron density map calculated from the low-energy dataset (contoured at 5 sigma) is indicative for cadmium and shown as unmarked mesh. The element specific double difference electron density maps are contoured at 7 sigma and are shown in grey (panel A, copper, panel B, zinc (marked Zn1)) mesh. The overall structure of APP-E2 is shown as cartoon representation; metal coordinating amino acid side chains are shown as sticks. The intramolecular binding sites M1 and M2 are highlighted in rectangles.

FIG. 11 Dependence of K_{D} on Tris concentration. K_{D} of copper to APP-E2 was measured at 15 mM, 25 mM, 50 mM and 75 mM Tris in ITC experiments. K_{Dapp} was plotted as function of [Tris], linearly fitted and evaluated assuming competition with Cu[Tris]₁ according to K_{Dapp}=K_{D}+K_{1Tris}×K_{D}×[Tris].

FIG. 12 Saturation of APP-E2 with zinc. (A) The ITC peak integrals of the first and third titration run of a serial titration experiment to achieve zinc saturation are shown as squares. Solid squares represent reliable data points whereas white squares mark data points adversely affected by dilution due to the refilling procedure necessary after each titration run. In addition to the exothermic binding reaction at low zinc/protein ratios, a continuously decreasing endothermic signal is generated, indicating several additional binding sites. At high zinc/protein rations the protein start to precipitate, reflected by the stable endothermic signal. (B) As the E2 domain tends to precipitate upon zinc saturation, the average number of zinc ions bound to the protein was determined experimentally. Initial precipitation by excess zinc followed by several wash steps resulted in a stable equilibrium ratio of zinc bound protein in the liquid phase and in the solid phase. Concentrations of zinc, protein and the ratio between zinc and protein are shown for seven subsequent wash steps, when the equilibrium was reached. The final ratio is 4.3 total (corresponding to one tight and 3.3 weak) zinc binding sites per E2 molecule.

### DESCRIPTION OF VARIOUS AND PREFERRED EMBODIMENTS

Based on the crystal structure of the amyloid precursor protein ectodomain 2 (APP-E2 domain) with a resolution of 2.0 Å, conformational changes of the domain could be identified. In the domain an intramolecular metal binding site (M1) that has a high specificity and affinity for zinc and copper was identified. In addition, several weaker metal binding sites were ascertained that are primarily stabilizing crystal contacts.

An isolated APP-E2 core domain, as used in the present context, refers to a core part of the APP-E2 domain and may be obtained by, e.g., proteolytic cleavage of a member of the highly conserved amyloid precursor protein gene family (generally referred to herein as APP molecules). A few charaterized APP molecules, which are generally provided in isolated and/or purified form are, e.g., the human APP₆₉₅ (APP Isoform APP₆₉₅ of Amyloid beta A4 protein (Fragment)−Length: 695) [SEQ ID NO: 10], other human APP splice forms such as APP₇₅₁ APP₇₇₀, and APP homologous such as mouse APP [SEQ ID NO: 11], APP Gallus gallus [SEQ ID NO: 12], APP Xenopus levis [SEQ ID NO: 13], APPa Danio rerio [SEQ ID NO: 14], APPL Drosophila melanogaster [SEQ ID NO: 15], APL1 Caenorhabditis elegans [SEQ ID NO: 16], human APLP1 [SEQ ID NO: 17] and APLP2 [SEQ ID NO: 18]. As the person skilled in the art knows, a core domain according to the invention can also be recombinantely produced using the widely available sequence data for amyloid precursor protein gene family members of various origin. The APP-E2 core domain according to the present invention is characterized by: (a) at least three alpha helices, namely the alpha B, alpha C, alpha D helix and optionally the alpha E and alpha F helix, wherein preferred embodiments include the alpha B, C, D and E helices or all five helices, and (b) a metal binding site (M1) to which the alpha B, C and D helices contribute and, optionally, a metal binding site (M2) to which the alpha D, E and F helices contribute. The APP-E2 core domain is able to bind transition metal ions such as copper and zinc. The alpha B helix is preferably 30 to 40, the alpha C helix is preferably between 50 and 60, the alpha D helix is preferably between 30 and 40, the alpha E helix is preferably 20 to 30 and the alpha F helix is preferably between 10 and 20 amino acids long. The regions between the helices are from 0 to 10 amino acids long. While some helices might be directly attached to each other, others are separated by up to 12 amino acids, including 1, 2, 3, 4, 5, 6, 7, 8, 9, 10 or 11 amino acids (more than 2 amino acids in this region are also referred to herein as “loops”). The APP-E2 domain contains all five helices.

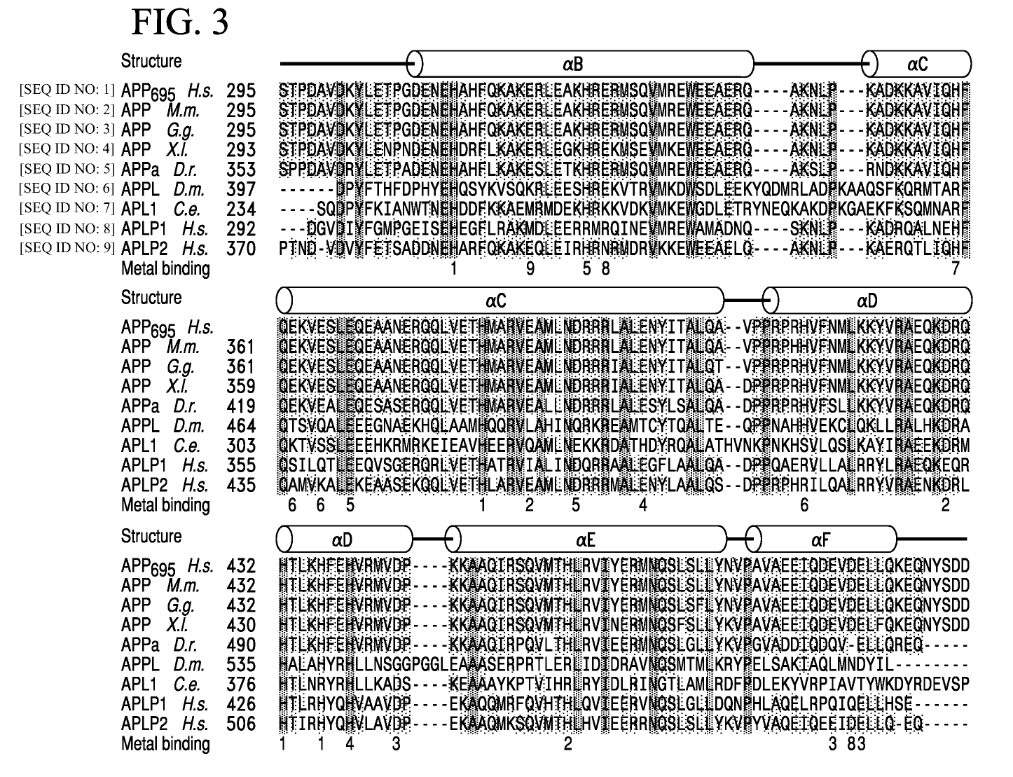

The APP-E2 core domain comprises amino acids which correspond to amino acids 301, 303, 312, 313, 334, 338, 349, 360, 361, 367, 368, 382, 385, 386, 388, 391, 395, 397, 404, 405, 410, 419, 424, 425, 428, 432, 439, 448, 459, 462, 467, 473, 477 of the human APP₆₉₅ (see also FIG. 3). The ability to bind a transition metal ion such as copper and zinc at the metal binding site M1 is mediated by 3 to 4 conserved histidine (His) residues in the alpha B, C and D helices. These 3 to 4 conserved histidine residues are, in human APP₆₉₅, amino acids 313, 382, 432 and 436 (the histidine at 313 is not used in the binding of zinc) and define the metal binding site M1. In certain embodiments, the metal binding site M2 is part of the APP-E2 core domain, which is formed by the amino acids glutamine (Glu), asparagine (Asp) and histidine (His) at amino acids residues corresponding to amino acid 387, 429 and 458 of the human APP₆₉₅. The term “corresponds to” or “corresponding to” makes clear that in other members of amyloid precursor protein family (APP molecules) while having the same overall structure, have the conserved amino acids at slightly different locations. For example, in the human amyloid beta (A4) precursor-like protein 1 (APLP1), the histidine that appears at amino acid residue 313 in human APP₆₉₅, appears at amino acid residue 308. Using, e.g., sequence alignments as those shown in FIG. 3, the appropriate residues can be readily identified in the corresponding APP family member.

GPC coupled static light scattering analysis confirmed a monomeric state of the present E2 domain in solution.

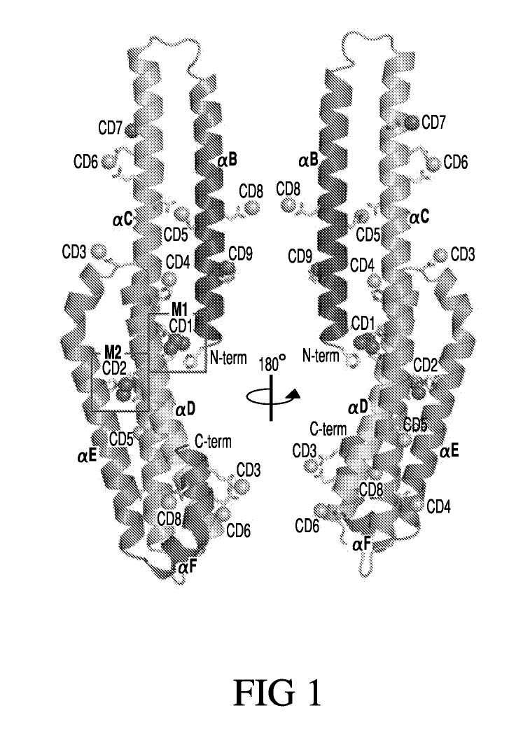

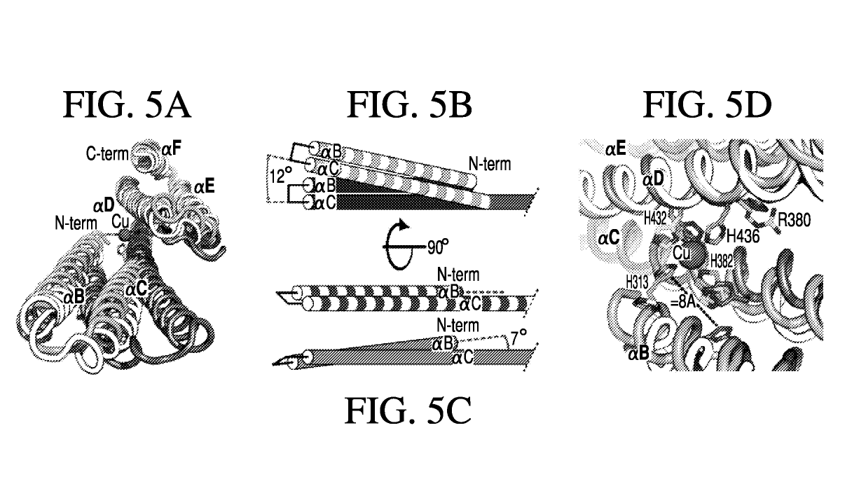

Conformation is, in the present context, the three dimensional structure of a protein molecule, in particular an APP molecule or a domain thereof, here in particular the APP-E2 core domain. In case of the APP-E2 core domain, the existence of its alpha helices which can move relative to one another is the common denominator of all physiological conformations. The term conformation as used herein conveys reversibility. Thus, if, in the present context, the transition from a first to a second conformation is described, this implies that a transition from the second conformation to the first conformation can take place when the original conditions are restored. As it is well known to the person skilled in the art, proteins and their domains such as the APP-E2 core domain are not static, but change in time and depending on the conditions to which they are exposed. An unrestricted conformation of the molecule or domain refers herein to a conformation in which the molecule or domain is purified, free of binding partners such a copper and zinc or other chemical entities that can change its conformation. An unrestricted conformation can be obtained by purifying, e.g., the APP molecule or domain thereof, in particular the APP-E2 core domain from chemical entities that influence its conformation, in particular metal ions. The domain can be simply purified and resuspended in, e.g., an appropriate buffer solution and/or certain ligands can be specifically removed, e.g., by using chelators. The restricted conformation does not describe a single conformation, but is one that differs from the unrestricted conformation and displays relative to the unrestricted conformation a reduced flexibility/increased rigidity of the core domain due to the binding of an agent. Since multiple restricted conformations exist, the invention includes, in particular when certain candidate agents are optimized, the transition from, e.g., a first restricted conformation to a second restricted conformation. The reduced flexibility or increased rigidity of the core domain is preferably a result of a rearrangement of the alpha B and alpha C helices, more in particular the alpha B helix and the N-terminal part of the alpha C helix (also as a unit referred to herein as N-terminal coiled coil, coiled coil or coiled coil like region or fold), wherein said N-terminal part of the alpha C helix is less than the full length helix, but comprises the histidine that is part of the metal binding fold (M1). This N-terminal coiled coil is preferably, to assume a restricted conformation, rotated as a whole by more than 5, 6, 7, 8, 9, 10, 11 or 12° around a hinge region in the alpha C helix which is located C-terminally of the histidine that is part of the metal binding site M1, wherein the rotation moves the histidine of at least the alpha B helix towards the histidines located at the N-terminal end of the alpha D helix. Thus the rotation is in plane with the alpha D helix and towards the same. Thus, as a result the histidine of the alpha B helix is closer to at least one of the other 2 histidines of the M1 metal binding site, which are located in the alpha D helix, preferably by at least 4, 5, 6, 7, or 8 Å. The rotation could, for example, for the representation on the left of FIG. 1, be described as a rotation of the coiled coil around the hinge point in the alpha C helix, wherein said hinge point is located proximal to the M1 metal binding site. Here the movement would be in the plane of the paper towards the right. An examplaratory movement is also illustrated in FIGS. 5 A and B. In FIG. 5B the lower horizontal coiled coil reflects an unrestricted conformation while the coiled coil that is moved out of this horizontal reflects a coiled coil in a restricted conformation. FIG. 5A shows a top view of both unrestricted (right) coiled coil and the restricted coiled coil (left). As can be seen from this depiction the restricted conformation is achieved by a rotation that moves the N-terminal part of the alpha B helix, which contains the M1 histidine, towards the center of the metal binding site, in particular towards the other 2 histidines of the M1 metal binding site, which are located in the alpha D helix.

A restricted conformation can be induced by the addition of a transition metal ion, in particular by the addition of zinc or copper. Whether a candidate agent according to the present invention results in a restricted conformation or can convert the restricted conformation into a unrestricted conformation can be measured via a wide variety of methods, including methods that detect the transition in conformation directly including, but not limited to, spectroscopic methods, such as circular dichronism (CD) or infrared (IR) spectroscopy and methods that detect the change indirectly such as nuclear magnetic resonance or by the determination of the melting temperature prior and after addition/binding of the candidate agent. In the latter case, an increase in melting temperature of a the APP-E2 domain or the APP molecule containing the same in a unrestricted conformation signifies that a transition to a restricted conformation was mediated by the candidate agent. An increase of more than 1, 2, 3, 4, 5, 6, 7, 8, 9 or 10° C. indicates that the candidate agent induced a conformational change that reduced the flexibility/increased the rigidity of the core domain. Other methods known to the person skilled in the art that can be used to follow the conformational change include, but are not limited to, measurement and analysis of delta G_{unfolding}, characterization of accessibility by e.g. limited proteolysis, characterization of flexibility by e.g. determination of hydrodynamic radius (e.g. gel permeation chromatography, dynamic light scattering) or a secondary assay that provides a readout that depends on the conformation of APP-E2 core domain.

In a certain embodiment of the invention helices, such as the alpha B helix and the alpha C helix are shifted out of alignment to each other. Thus, the helices, e.g., alpha B helix and the alpha C helix which were previously parallel display an angle relative to each other of at least 4°, 5°, 6° or 7°. This embodiment mimics the conformational shift resulting from the binding of a copper ion to the metal binding site M1, which is defined by the 4 histidines as described above. When zinc is bound, the alignment of the two helices remains parallel. The term parallel as used in the present context is not a strict mathematical term, but a biological term and includes misalignment not exceeding the helices having an angle towards each other of up to 1.5°.

The term “conformation shifting operation” refers to an operation that utilizes the structure coordinates of a chemical entity and the APP-E2 core domain, more in particular structure coordinates of the APP-E2 core domain in or around the metal binding site M1 or at least one amino acid residue on the surface (which includes the metal binding site) in or around M1 of the APP-E2 core domain that is not more than, 40, 30, 20, or 10 Å away from any one of the histidines of M1. This operation may be achieved by positioning, rotating or translating a chemical entity in the metal binding site to match the shape and electrostatic complementarity of the metal binding site or by positioning, rotating or translating the chemical entity relative to at least one amino acid residue on the surface of the APP-E2 core domain that is preferably not more than 40, 30, 20, or 10 Å away from any one of the histidines of the metal binding site. Covalent interactions, non-covalent interactions such as hydrogen bond, electrostatic, hydrophobic, van der Waals interactions, and non-complementary electrostatic interactions such as repulsive charge-charge, dipole-dipole and charge-dipole interactions may be optimized (see also U.S. Pat. No. 7,809,541).

The term “generating a three-dimensional structure” or “generating a three-dimensional representation” refers to converting the lists of structure coordinates into structural models or graphical representation in three-dimensional space. This can be achieved through commercially or publicly available software. The three-dimensional structure may be displayed or used to perform computer modeling, in particular conformation shifting operations. In addition, the structure coordinates themselves may be used to perform computer modeling and conformation shifting operations.

In the crystal structure of the E2 domain of APP₆₉₅ it was observed that the four highly conserved histidine side chains at metal binding site M1 selectively provide a coordination geometry that fits to the first row transition metals copper and zinc, whereas the larger cadmium ion adopts a multiple occupied state. Here, coordination of zinc was facilitated by three histidine residues and a water molecule, replacing the side chain of His313 (human APP 695) as binding partner. The pH-dependent zinc binding properties of full length APP (Bush et al., 1993) are in excellent agreement with a histidine-mediated coordination. Comparable to catalytic zinc sites of alpha class carboanhydrases (Liljas et al., 1972) and proteases of the metzincin-family (Bode et al., 1993), a distorted tetrahedral ligand assembly was observed in the E2 domain structure. The solvent accessibility of the zinc bound water molecule and the charged cleft harboring the metal binding site was indeed reminiscent of a metalloprotease; however the required catalytic glutamate residue is missing. In competition to zinc binding, four histidine side chains of the E2 domain contribute to copper coordination at this site, including said His313.

The functional importance of the metal binding site of the APP-E2 domain is evident from the large conformational changes of the protein confirmed by, e.g., increased thermostability and the altered protease accessibility upon binding of copper and zinc. The metal free but not the metal bound state of the E2 domain allows bending of the central elongated helix and reorientation of the N-terminal coiled coil region. A rather rigid conformation of the C-terminal helix bundle was shown in absence of the coiled coil by NMR studies (Dulubova et al., 2004). Binding of copper and zinc to APP-E2 did not only prevent structural flexibility but rather stabilized distinct conformations that depend on the exact coordination sphere and hence result in specific biochemical properties. Copper binding by the E2 domain comprised cooperative action of four sequentially distant histidine side chains stabilizing the overall structure, whereas the zinc bound state affected particularly the central helix and the C-terminal helix bundle. These characteristics suggest a metal ion triggered conformational switch that converts the protein into different functional states, in principle similar to metal-dependent regulatory proteins like calmodulin (Gifford et al., 2007). In contrast to the binary on/off switch of calmodulin, the competitive metal binding properties of the E2 domain implicate regulation in an even more complex manner, employing multiple functional states.

Several facts strongly suggest that the described competitive binding of Zn²⁺ and Cu²⁺ as well as the resulting conformational changes are of high physiologic relevance. Membrane bound and soluble APP are transported to and localized at synaptic terminals (e.g. (Groemer et al., 2011; Hoey et al., 2009; Sabo et al., 2003; Schubert et al., 1991)). Maximum concentrations of more than 10 μM zinc (Paoletti et al., 2009) and/or copper (Hung et al., 2010) are reached during neuronal activity in hippocampal glutamatergic synapses. These values are elevated by several orders of magnitude in comparison to the basal levels of ˜20 nM free zinc and ˜50 nM free copper, measured in the brain interstitium (Frederickson et al., 2006) and in the cerebrospinal fluid (CSF) (Boll et al., 2008), respectively. A dissociation constant of the E2/zinc complex of lower than 4 μM was estimated, which is in the same order of magnitude as previously reported for full length APP (0.75 μM) (Bush et al., 1993), indicating a significant affinity for zinc within its physiological environment. Interestingly, Cu²⁺ binds tighter to APP-E2, again in excellent agreement with the biochemical properties of full length APP (10 nM) (Hesse et al., 1994). Already an underestimated correction for the competition with the buffer compound (Hatcher et al., 2008) resulted in an absolute K_{D} of lower than 13 nM. Correspondingly, copper displaces zinc from the protein, a competition also verified in respective ITC experiments. In consequence, the E2 domain of APP sensitively responds to elevated copper levels even in the presence of zinc, implicating an antagonistic action of the metal bound states also in the cellular context. Sequence alignments reveal a high degree of conservation of this metal binding site among APP family proteins, including APLP1 and APLP2, which are generally referred to herein as APP molecules. This indicates its central function in APP like proteins and them following a similar antagonistic copper- and zinc-dependent regulation scheme reflecting the functional redundancy described for the APP family members (Anliker and Muller, 2006). Consequently, misregulation of the E2 domain based on imbalanced levels of copper and zinc is expected to impair the proper function of APP and APP like proteins.

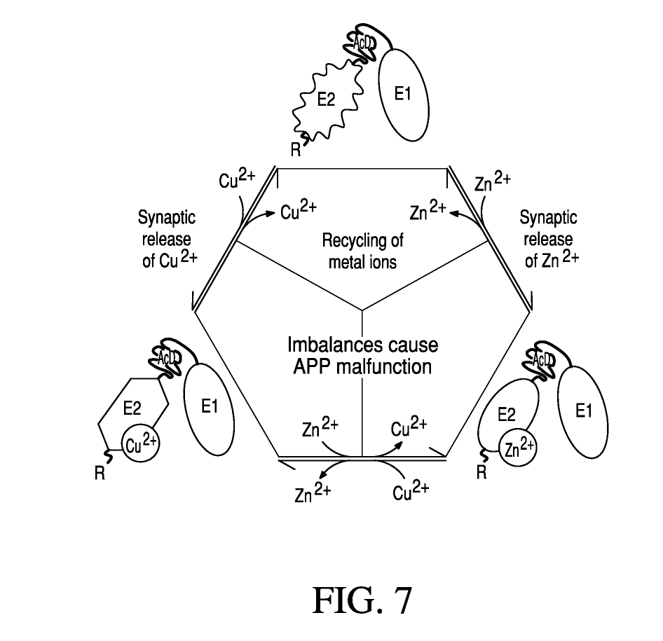

The model of regulation of APP molecules is shown in FIG. 7:

Metal binding specifically switches the conformation of the E2 domain, which in turn modulates the interaction with its binding partners and finally the trafficking of APP molecules (APP is shown as an example). This regulation scheme not only applies to the depicted APP, but also applies to the E2 domains of APLP1 and APLP2. Both proteins contain the metal binding residues and are capable to interact with F-Spondin (Ho and Sudhof, 2004). Dysregulation of the neuronal metal homeostasis in AD therefore interferes with the regulation of all APP-family proteins (APP molecules). Malfunction of APP molecules such as APP, APLP1 and APLP2 may therefore directly contribute to the pathology in AD.

The excellent diffracting crystals obtained enable a structure guided approach to, e.g., develop and select agents that specifically interfere with the metal-dependent conformation of the E2-domain.

Candidate agents according to the present invention include peptides comprising at least 6 amino acids, proteins, antibodies, anticalines, functional nucleic acids (aptamers, aptazymes, ribozymes, spiegelmers, antisense oligos, si RNA), natural compounds and small molecules or combinations thereof. Small molecules have, in the present context, a molecular weight of between 50 and 1000 g/mol, preferentially between 150 and 800 g/mol. Peptides, proteins and/or antibodies of the present invention comprise proteinogenic- and/or non-proteinogenic and/or natural and/or non-natural amino acids. They can be chemically modified by naturally occurring or artificial modifications of the constituting amino acids, including but are not limited to, acetylation, carboxylation, farnesylation, phosphorylation and ubiquitinylation. In certain embodiments, proteins known to physically interact with APP molecules are optimized. Targets of optimization include, but not limited to, F-spondin (Ho & Sudhof, PNAS, 101 (2004) 2548-53) or SorLA (Anderson et al., Biochemistry, 45 (2006) 2618-28), or domains, parts, modifications or substitutes derived therefrom. Antibodies known to interact with APP molecules may also be optimized. Those include, but not limited to Anti-5 and 1G5 (Mattson, et al., Neuron, 10 (1993) 243-254), or domains, parts, antigenic sequences or regions (CDAs), modifications or substitutes derived therefrom. However, any chemical entity that might induce the above mentioned conformational change in said APP core domain can be employed in the present invention. Such a compound might or might not contain a metal such as copper or zinc and might or might not directly interact with one of the metal binding sites. Candiate agents may be contained in a library of compounds and/or may have been preselected by, e.g., screening in-silico compound libraries using the three dimensional structure of the APP-E2 core domain. During this screening procedure, chemical entities that may influence the conformation of the APP-E2 core domain are selected from the library. Suitable libraries are, for example available on the BIOSCREENING website which can be found online under the respective dot com extension. Alternatively, the candidate agent can be selected by performing a computer assisted design, selecting candidate agents from the library of compounds by providing the three dimensional structure of at least the APP-E2 core domain and testing chemical entities for APP-E2 core domain interactions such as electrostatics, H-bonding interactions, hydrophobic interactions, conformational strain and/or size requirements. In a preferred embodiment, the candidate agents bind no more than 30 Å, 20 Å or 10 Å away from the metal binding site M1.

From the pool of candidate agents, agents meeting the set criteria are selected and can be, as the person skilled in the art will appreciate, further evaluated by in vitro enzymatic or cell based assays as well as in vivo methods.

Suitable in vitro methods include enzymatic and/or cell based assays based on sectretase activity, in particular alpha and/or beta sectretase activity. Suitable enzymatic assays include the BACE FRET assay or the Human alpha-secretase ELISA Kit from EIAab & USCNLIFE (Wuhan EIAab Science Co., Ltd). Suitable cell based assays include alpha and/or beta sectretase cell based assays such as BACE Cell Based Assay (see also US Patent Pub. 2010/0222338).

Computer implemented methods generally involve defining a binding region. This defined binding region is used to narrow down the pool of chemical entities. Preferably this binding region according to the present invention is the metal binding site M1 and residues surrounding M1, preferably residues on the surface of a APP-E2 core domain which are located within 30 Å, 20 Å or 10 Å of M1 and are therefore likely to interact with an agent of interest. Candidate agents may be selected that either directly interfere with metal binding at M1 or stabilize, e.g., the interaction between helices alpha C and alpha D close to their intersection point and thus close to or at M1. Candidate agents that have allosteric effects bind more remotely and are preferably larger (e.g., antibodies) and, e.g., simultaneously bind to the loops between helices B and C and between helices D and E.

### High Resolution X-Ray Structure of the Human APP-E2 Domain.

A new tetragonal crystal form that grew from cadmium containing crystallization buffer and diffracted X-rays to a resolution of 2.0 Å was identified. Initial attempts to solve the phase problem by molecular replacement using the available structure of human APP-E2 (PDB-id: 1RW6, (Wang and Ha, 2004)) failed, implicating major conformational differences (see below). Using the truncated C. elegans APL-1 E2 structure (PDB-id: 3K66; (Hoopes et al., 2010)) a partial solution was obtained. Protein bound cadmium atoms were identified from anomalous difference Fourier maps. Final phases were calculated by single anomalous dispersion (SAD) resulting in a well defined experimental electron density map. The structure was build and refined to 2.0 Å resolution, showing good R-factors and stereochemistry (Table I). Exclusively composed of α-helical secondary structure, the molecule adopts an elongated shape spanning ˜95×35×25 A³ (FIG. 1). The fold is dominated by the central, ˜90 Å long (15 helical turns) helix α-C, which is in contact to all other helices. Helix α-B (e.g., His313-Gln344 in human APP₆₉₅, which has been selected herein for further evaluation) and the first 36 amino acids of the central helix α-C (e.g., Lys350-Arg385) contribute to a coiled-coil like fold (hereinafter also “coiled coil”). The C-terminal part of helix α-C (e.g., Val386-Gln406) together with the helices α-D (e.g., Arg411-Asp444), α-E (e.g., Lys446-Tyr474) and α-F (e.g., Val479-Gln492) constitute a four helix bundle enclosing a small hydrophobic core. Compared to similar structural arrangements e.g. in alpha spectrin (Grum et al., 1999) the strict heptad repeat pattern is distorted in the E2 N-terminal coiled coil like region as noted before (Wang and Ha, 2004). Analysis of the contact surfaces of those helices for different E2 structures (Table V) revealed a similar solvation free energy gain (ΔᵢG) (Krissinel and Henrick, 2007) of the interaction. Hence, different relative orientations of these helices result in similar hydrophobic inter-helix contacts, explaining the high flexibility observed in previous structural studies of the human E2 domain (Dulubova et al., 2004; Keil et al., 2004; Lee et al., 2011; Wang and Ha, 2004).

### Human APP-E2 is Monomeric.

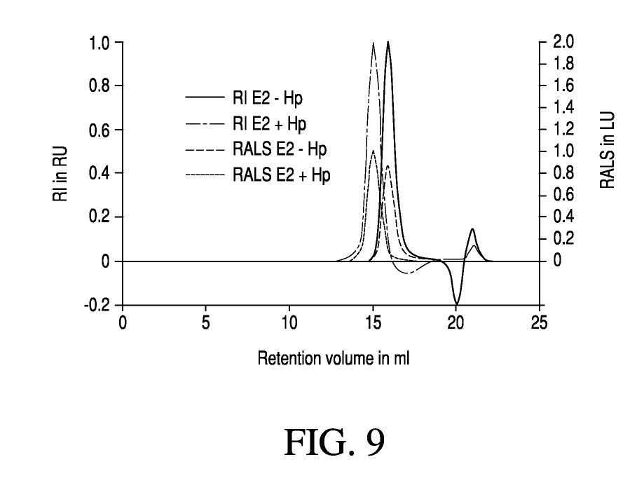

All intermolecular contacts identified in the crystals analysed with the PISA server (Krissinel and Henrick, 2007) result from crystal packing, evidenced by the calculated complexation significance scores (CSS) that are equal to 0. Consequently, the protomer contacts are predominantly stabilized by the intermolecular coordinated cadmium ions. Dimeric assemblies of the E2 domain related by a proper 2-fold symmetry (Lee et al., 2011; Wang and Ha, 2004) do not exist in our crystal form, disfavoring their presence in solution. To investigate this observation further, the apparent molecular weight was determined based on the hydrodynamic radius (MWᵣₕ) using gel permeation chromatography (GPC) as well as the absolute molecular weight (MW_{SLS}) employing GPC coupled static light scattering (SLS; Supplementary Figure S2). Both values (Table II, first row) are in good agreement with the theoretical molecular weight of the protein (25.6 kDa), indicating that APP-E2 is monomeric in solution, comparable to the C. elegans homologue APL-1 (Hoopes et al., 2010). The influence of heparin on the oligomerization state of the E2 domain, which was described to promote dimer formation (Lee et al., 2011), was tested. Addition of the low molecular weight heparin Clivarin to the GPC buffer resulted in an increased apparent molecular weight of 39.2 kDa (indicating an increase in the hydrodynamic radius) but in a very low increase in the absolute molecular weight to 31.6 kDa (SLS data), matching monomeric E2 (Table II, FIG. 9). These data rather indicate binding of one heparin molecule of around 4 kDa to one protein molecule, possibly adding flexibility or leading to a change in the overall molecular shape of the complex that accounts for the increased hydrodynamic radius.

### Conserved Intramolecular Copper/Zinc Binding Site in APP-E2.

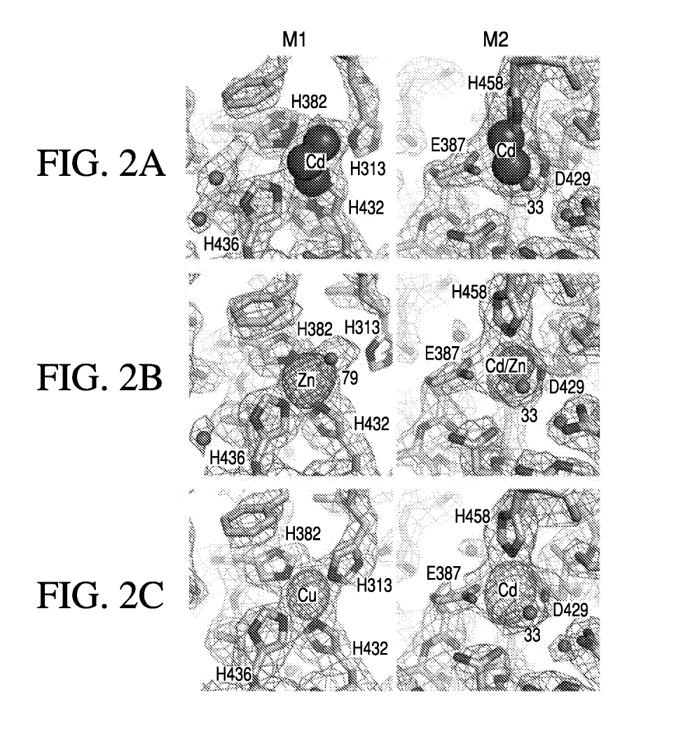

Interestingly, Cd²⁺ ions present in the crystallization buffer bound at various intermolecular (Cd3, Cd4, Cd5, Cd6, Cd8) and intramolecular sites (Cd1, Cd2, Cd7, Cd9) (FIG. 1). Most interestingly, the cadmium ion Cd1 at metal binding site M1 is bound by four histidine sidechains (His313, 382, 432 and 436) (FIG. 2A) in a multiple occupied state cross-connecting helices α-B, α-C and α-D. Similarly, helices α-C, α-D and α-E contribute to the multiple occupied state of Cd2 bound by Glu387, Asp429, His458 and a defined water-molecule at metal binding site M2. Cd7 and Cd9 are bound by one amino acid side chain only, corresponding to weak binding sites that show a low occupancy in the structure. In consequence both, the E2 domain and the crystal lattice, are stabilized by a large number of defined Cd²⁺ ions, resulting in reduced flexibility of the domain and high diffraction power of this crystal form.

The metal binding sites M1 and M2 provide coordination spheres suited for intramolecular binding of physiological transition metal ions like copper and zinc. To probe the competitive displacement of Cd²⁺, two complete datasets of crystals soaked in a solution supplemented with either Cu²⁺ or Zn²⁺ in addition to Cd²⁺ were collected in the vicinity of the K-absorption edge for each of the two elements (Table I) in order to calculate element specific anomalous double difference electron density maps (Than et al., 2005) (FIG. 2).

Zn²⁺ replaces Cd²⁺ and is coordinated by three histidines (His382, 432 and 436) as well as one water molecule in an almost tetrahedral geometry (FIG. 2B). The coordination sphere at this site changes if Cd²⁺ is replaced by Cu²⁺ and the electron density of His313 becomes well defined. Now four histidines form a coordination sphere are best described as tetrahedrally distorted square plain (FIG. 2C). At metal binding site M2, the bound cadmium ion Cd2 was only partially replaced by Zn²⁺ and not affected by Cu²⁺ in the soaked crystals, indicating less specificity of M2 for Zn²⁺ and Cu²⁺ compared to site M1 (FIG. 2A-C). The conservation analysis of both internal metal binding sites is in excellent agreement with this observation. Alignments of APP, APLP1 and APLP2 sequences show some, but not complete conservation of the amino acids at site M2 (FIG. 3). However, residues His313, 382, 432 and 436 of APP₆₉₅, shown to specifically bind copper and zinc ions, are highly conserved in the APP-superfamily, amongst others in APLP1 and APLP2 (FIG. 3).

### Binding of Copper/Zinc in Solution.

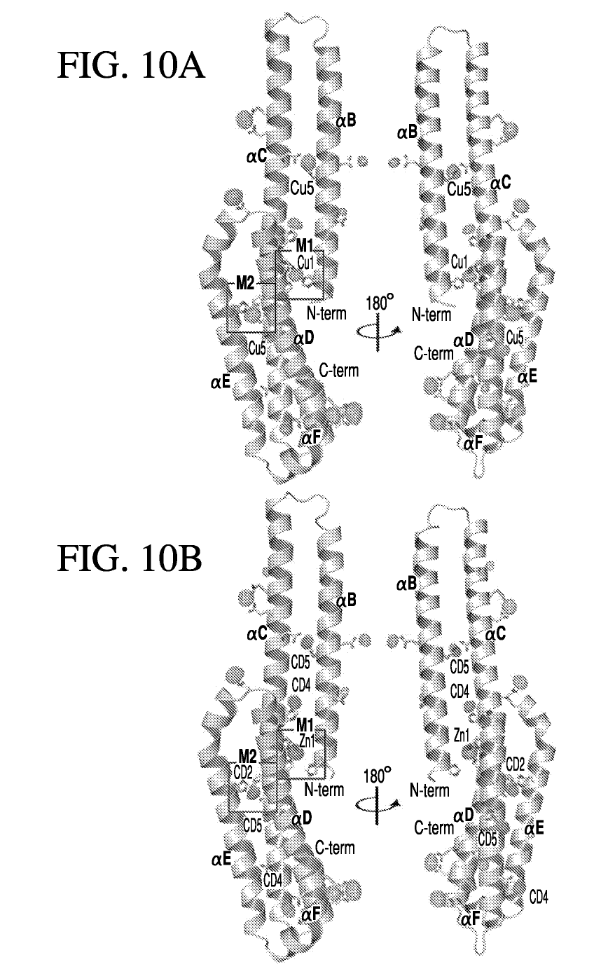

Several of the intermolecular metal binding sites can at least be partially occupied by copper or zinc ions, as observed in the crystal soaking experiments (FIG. 10). To avoid these intermolecular contacts and hence precipitation of the E2 domain in solution, 50 mM Tris was used as buffer to complex Cu²⁺.

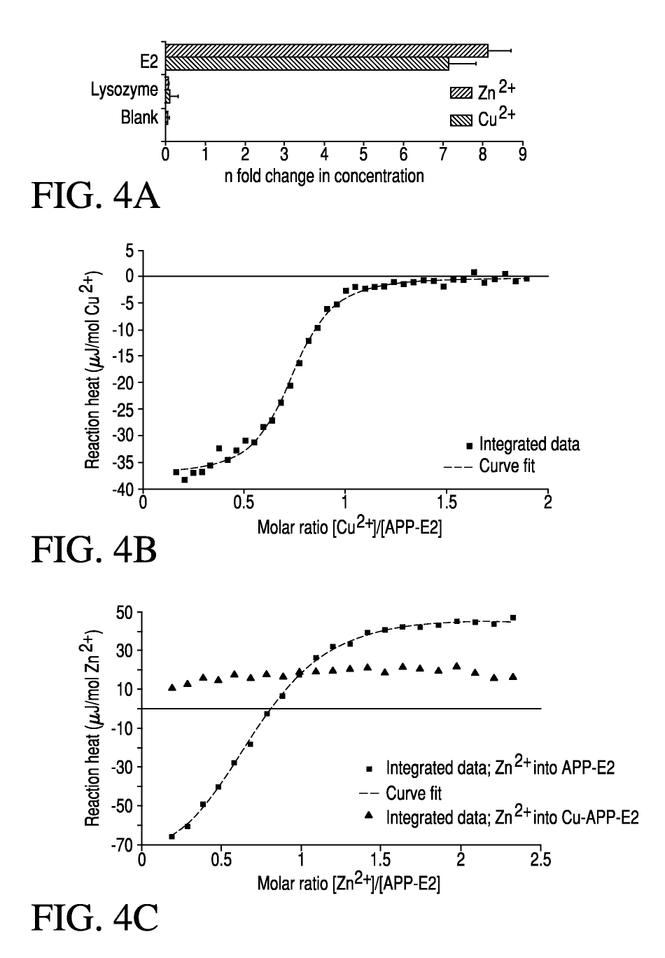

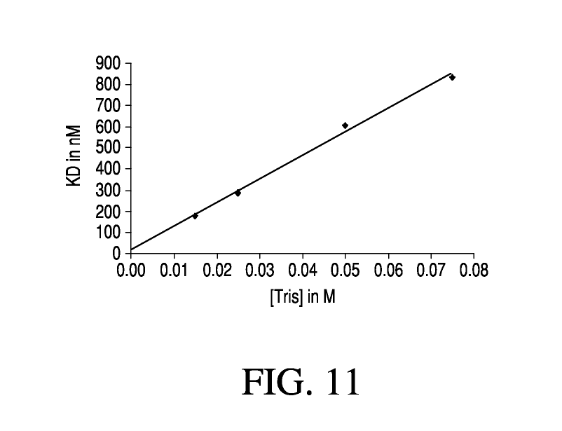

The ability of the E2 domain to bind copper and zinc in solution was confirmed in an ultrafiltration-based retention assay (FIG. 4A). Both, copper and zinc ions accumulated in the retenate of the ultrafiltration device upon APP-E2 concentration. In the presence of lysozyme, not capable of binding these metals and in the absence of any protein, neither copper nor zinc was concentrated. Determination of the binding strength by isothermal titration calorimetry (ITC) revealed tight binding of copper to the E2 domain (FIG. 4B), characterized by an apparent dissociation constant K_{Dapp} of 610 nM. This apparent dissociation constant has to be corrected, taking into account competition by the ligand Tris and its pH-dependent protonation (Table III). Estimating a minimal model of a Cu²⁺-Tris 1:1 complex, a binding constant K_{D} of 13 nM was calculated as upper (weakest) limit (see Materials and Methods for details). In fact, our determination of apparent K_{D} as function of the Tris concentration followed by a linear extrapolation yields a K_{D} of ˜20 nM as intersection point with the ordinate (FIG. 11). A similar calculation assuming the quinary Cu(Tris)₄-complex (Hatcher et al., 2008) results in values as low as 2 μM.

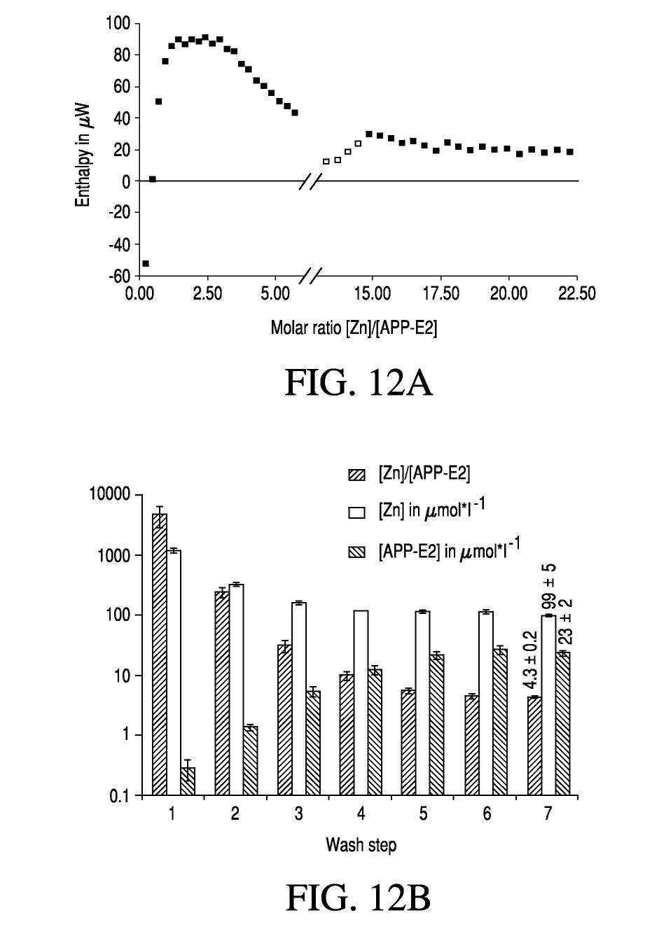

In ITC experiments to study the formation of the Zn/APP-E2 complex, the addition of specific zinc chelators to the Hepes buffer resulted in apparent binding constants that were too low for numeric evaluation. Therefore, measured zinc binding in the absence of a zinc-specific chelator was measured and the ITC data was evaluated as arising from multiple zinc-binding sites including unspecific association of zinc ions to the protein surface. The binding isotherm showed an initial exothermic reaction corresponding to one strong interaction followed by an endothermic contribution apparently arising from multiple weak binding sites (FIG. 4C). ITC experiments at high zinc to protein ratios (FIG. 12A) showed a continuous, smooth decline of the endothermic reaction. At zinc to protein ratios exceeding 5:1, the protein begun to precipitate. The stiochiometry of these weak, non-saturable binding sites was determined experimentally and used to evaluate the binding curves employing a model of two sets of independent binding sites (materials and methods, FIG. 12B). The resulting dissociation constants K_{D1} of 3.9 μM and K_{D2} of 2.6 mM have to be regarded as upper (weakest) limit since K_{D2} is determined not statistically significant in the fit (Table III). A possibly lower K_{D2} necessarily results in a decreased K_{D1} value. Higher values of K_{D2} however would affect K_{D1} slightly, but would cause an increase of ΔH₂ to values even higher than 1200 kJ/mol, unlikely for non-covalent interactions.

To verify that zinc and copper compete for the same binding site in solution, the zinc-binding experiments were repeated with APP-E2 preparations pre-saturated with copper. If copper ions are bound to the protein, the exothermic reaction upon Zn²⁺ addition is completely missing from the ITC-profile, as expected for a competitive metal binding site (FIG. 4C). In contrast, the unspecific binding is still observed as indicated by the continuous endothermic signal.

### Conformational Differences Between Metal-Bound and Metal-Free Structures of the E2 Domain.

A structural alignment of the C-terminal parts (e.g., Ala388-Gln492 of human APP₆₉₅) of the copper bound (this work) and the metal-free human APP-E2 structure (Wang and Ha, 2004) (PDB-ID: 1RW6) revealed a specific reorientation of the N-terminal segment (FIG. 5A). In the metal free protein, helix α-B and the N-terminal part of helix α-C are highly flexible and can adopt different conformations. Conformational dynamics of this segment were confirmed in APL-1 (Hoopes et al., 2010) and APLP1(Lee et al., 2011) underlining a general mode of flexibility of the E2 domains in the APP-superfamily. Coordination of metal ions requires a specific geometry and hence forces the whole E2 domain in a specific conformation. The N-terminal coiled coil, here consisting of the N-terminal half of helix α-C and the preceding helix α-B, was rotated as rigid body by 12° (Angle of alpha-carbon segments between Ala355-Ala388 of human APP₆₉₅) (FIG. 5B). Rotation occurs, in this instance, at a hinge region between His382 and Ala388. An additional movement twists the helices α-B and α-C around each other by about 7° in the metal bound form (FIG. 5C). His313 at the N-terminus of helix α-B relocates more than 8 Å towards the metal binding center (FIG. 5D).

Coordination of cadmium and especially of copper requires the rearrangements observed in the respective E2 structures and the associated proper orientation of the four coordinating histidine side chains (e.g., His313, 382, 432 and 436 in human APP₆₉₅). In contrast, no specific orientation of the N-terminal helix should be required in the zinc bound state as His 313 is replaced by a water molecule. In the crystals produced the conformation of the cadmium bound form was “trapped” as they were grown in presence of this ion. Even if the metal at binding site M1 becomes exchanged to Zn²⁺ later on, movement of the N-terminal helix seems to be still restricted by the crystal packing.

The comparison to the conformation of the APL-1 structure shows that replacement of His436 by Arg380 in the C. elegans APP homologue protein apparently causes a metal-independent conformational shift. Due to sterical reasons, the bulky arginine side chain forces helix α-C of the APL-1 E2 domain in an orientation similar to the human metal bound E2 structures. However, the N-terminal helix α-B of the APL-1 E2 domain is not fixed, similar to the conformation expected for the solely zinc bound (but not for the copper and cadmium bound) human protein. Correspondingly, the twist of the helices α-B and α-C, essential to facilitate the coordination of copper and cadmium by the human E2 domain, is not observed in the APL-1 E2 structure. Hence, the APL-1 E2 domain seems to represent a structural intermediate in between the copper bound and metal free state of the human protein (FIG. 5A-D), as expected for zinc bound human APP-E2.

Consequently, the available structural data imply at least 3 different conformational states of the APP-E2 domain, differing in the flexibility of the N-terminal coiled coil: In the metal free protein, helix α-B and the N-terminal part of helix α-C are highly flexible and can adopt different conformations. If zinc is bound, a partial movement of the amino acids located N-terminal of coordinating His382 corresponding to human APP₆₉₅ is still possible. However, binding of copper requires concerted structural rearrangements to establish the required coordination sphere and hence movement of the coiled coil helices is restricted.

As the person skilled in the art will readily appreciate other restricted conformations are within the scope of the present invention as is a conformational switch from a first restricted conformation to a second restricted conformation and vice versa. Thus, both for computer implemented methods as well as in vitro methods chemical entities/candidate agents may be selected that are known to bind to APP molecules. A binding to the APP-E2 core domain can be verified via the methods disclosed herein. Additionally or alternatively, this chemical entity/candidate agent might be modified to alter its binding to the APP-E2 core domain. This modification might induce, e.g., an alternation in said candidate agents conformation from a first restricted conformation to a second restricted conformation, from a second restricted conformation to a first restricted conformation or from a restricted conformation to an unrestricted conformation.

### Binding of Copper/Zinc Stabilizes Metal-Dependent Conformations of the App-E2 Domain in Solution.

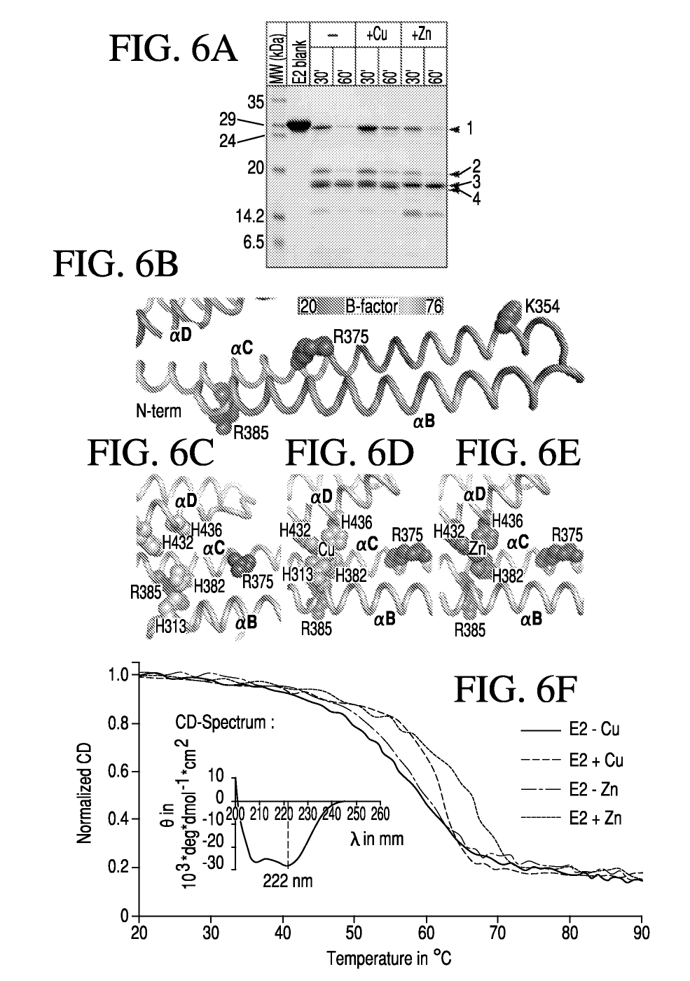

Dulubova et al. (Dulubova et al., 2004) identified a stable fragment in limited proteolysis experiments with Trypsin, comprising the amino acids Val385-Arg501 of APP₆₉₅. Interestingly, the N-terminal boundary exactly matches the hinge region identified in the E2 domain (see above) and lacks a major part of the central helix α-C and helix α-B, including the metal coordinating residues His382 and His313. As binding of metal ions is expected to affect the flexibility of helices α-C and α-B and consequently the susceptibility to Trypsin cleavage, limited proteolysis experiments were performed (FIG. 6A). The N-termini of the resulting peptide fragments were determined by Edman sequencing (Table IV). The major Trypsin cleavage products of human APP₆₉₅ in absence of any metal ions indeed indicated rapid degradation of helices α-C and α-B at Lys354 (fragment 2), Arg375 (fragment 3) and Arg385 (fragment 4). In addition, our structure shows elevated B-factors of the amino acids N-terminal of Ala355, indicating the inherent flexibility of the N-terminal region (FIG. 6B). In contrast, the remaining C-terminal fragment starting at Val386 remains stable for more than one hour in the presence of Trypsin once the N-terminal coiled coil region is cleaved off.

Addition of copper to the proteolysis reaction stabilizes especially fragment 1, corresponding to the full-length protein, and to a lesser extent also fragments 2 and 3 (FIG. 6A). This observation is in excellent agreement with the structural properties of the copper bound E2 domain: Coordination of copper requires a specific orientation of His313, His382, His432 and His 436 and hence a proper orientation of the helices α-B, α-C and α-D. Vice versa, helices α-B, α-C and α-D are cross connected and their movement is necessarily restricted upon copper binding, reducing the overall flexibility and preventing the protein from proteolytic attack (FIG. 6C, D). Once the N-terminal helix is cleaved off by the protease, only three histidine side chains are left for copper coordination, which is expected to weaken the binding significantly. Accordingly the stabilizing effect of copper is expected to be weaker for the smaller fragments 2 and 3 compared to fragment 1 as seen in FIG. 6A.

Due to the different coordination sphere seen for APP-E2 bound Zn²⁺, not involving His313 of helix α-B, a different effect of this ion on APP-E2 stability was expected. Indeed, when Zn²⁺ was present in the cleavage reactions, a very strong site specific protection was observed. Trypsin cleavage at Arg385 completely disappeared and fragment 3 accumulated (FIG. 6A) and proved stable for more than one hour. The abundance of the N-terminal fragment 5 also increased. Apparently zinc specifically stabilizes the region between Arg375 and Arg385 including the coordinating His382 of APP₆₉₅. It does, however, still allow flexibility in the region N-terminal of Arg375 as His313 at the N-terminus of helix α-B does not contribute to Zn²⁺ binding (FIG. 6E).

In agreement with the limited proteolysis data a metal-dependent melting behavior of the APP-E2 domain by CD-spectroscopy was observed (FIG. 6F). Addition of copper and zinc results in an increased CD-signal at 222 nm and hence increased alpha helical content of the protein at high temperatures. The zinc-bound E2 domain unfolds continuously in between 50° C. and 70° C. and the melting temperature increased from 57.8±0.3° C. to 64.2±0.3° C. upon addition of 10 μM Zn²⁺. Copper binding to the E2 domain is accompanied by a largely altered unfolding behavior of the protein. In addition to the elevated melting temperature (57.3±0.3° C. vs. 61.0±0.0° C. in plain buffer and after addition of 10 μM Cu²⁺, respectively), an increased unfolding cooperativity is observed in presence of copper. The protein collapses at temperatures above 61° C., indicated by an increased slope of the melting curve. The structural data give a good explanation for this cooperative collapse: The N-terminal coiled coil is characterized by increased flexibility and is consequently expected to be more sensitive to heat induced unfolding than the C-terminal helix bundle. Copper binding stabilizes the protein in general, but destabilization or even partial unfolding of the N-terminus at elevated temperatures will weaken the coordination of Cu²⁺ by His313 corresponding to APP₆₉₅. Once this bond breaks, the affinity to copper decreases drastically, resulting in higher flexibility and rapid unfolding of helix α-B, which in turn triggers unfolding of the entire E2 domain. In contrast, zinc binding causes a local stabilization of helix α-C and the C-terminal core structure. It still allows flexibility of the N-terminal coiled coil region and does not result in increased cooperativity during unfolding.

### Materials and Methods

### Expression and Purification.

APP-E2 comprising Ser295-Asp500 (numbering according to APP₆₉₅) was recombinantly expressed in E. coli BL21(DE3)pRIL and purified by immobilized metal ion chromatography (IMAC) and gel permeation chromatography (GPC) as described (Keil et al., 2004). For crystallographic and biochemical studies the C-terminal hexa-histidine tag was cleaved either with 20 U/mg protein coagulation factor Xa (Novagen) for 24 h at 20° C. or 1.5 μg/mg protein V8-protease (Calbiochem) for 1 h at 25° C., respectively. Protease and uncleaved protein were removed by application to either a Benzamidine sepharose 4 FF column (GE Healthcare) combined with a HisTrap FF Crude column (GE Healthcare) in case of factor Xa, or to a HisTrap FF Crude column combined with a HiTrap Heparin HP column (GE Healthcare) in case of V8-protease.

### Crystallization and Structure Determination.

Diffraction quality crystals were grown by the sitting drop vapor diffusion method at 20° C., mixing equal volumes of 10 mg/ml protein solution and reservoir containing 0.1 mM HEPES, pH 6.4, 1 M sodium acetate, 10 mM MgCl₂ and 50 mM CdSO₄; 12.5% R(−)-2-Methyl-2,4-pentanediol was added for cryo-protection. In competitive metal binding experiments crystals were soaked in reservoir solution complemented with 10 mM CuCl₂ or ZnCl₂ and 40 mM CdSO₄ for 12 h, including the cryoprotectant. Datasets were collected at 100 K at the synchrotron (BESSY/BL14.1/Berlin). A high resolution dataset extending up to 2.0 Å and a highly redundant SAD dataset extending to 2.7 Å at low energy was measured using cadmium containing crystals. To prove Cu²⁺ and Zn²⁺ binding, the exact energetic location of the corresponding K-absorption edges was determined via X-ray fluorescence scans and complete anomalous datasets were collected at either end of the respective K-absorption edge. Crystallographic data were processed with programs of the CCP4 suite (Project, 1994) (Table I). The phase problem was solved by a combination of MR and SAD. The PDB-structure 3k66 (Hoopes et al., 2010) (APL1; C. elegans) was modified with chainsaw (Stein, 2008) and MR was calculated in PHASER (McCoy et al., 2007). Applying these phases to the cadmium SAD low energy dataset, seven cadmium sites were identified, followed by SAD phasing and density modification in SHARP (de La Fortelle and Bricogne, 1997). The initial model, build in buccaneer (Cowtan, 2006), was transferred to the 2.0 Å resolved cadmium dataset as well as to the copper and zinc peak datasets, manually revised in MAIN (Turk, 1992) and refined in CNS v1.3 (Brunger, 2007)) (for details see Table I). All main chain angles fall into the most favored and additionally allowed regions of the Ramachandran plot (Ramachandran and Sasisekharan, 1968). Bond length, dihedral angles and bond angles of His313, 382, 432 and 436 to Cd²⁺, Zn²⁺ and Cu²⁺ were restrained to corresponding average values derived from the Cambridge Structural Database (CSD) (Allen, 2002). Composite annealed omit maps were calculated in CNS v1.3 omitting 2.5% of the final model. Element-specific ‘difference DANO maps’ were calculated according to (Than et al., 2005). PYMOL (DeLano Scientific LLC, USA, www.pymol.org) was used for molecular graphics and the sequence based structure alignments of metal bound APP-E2 (R385-L490), metal free APP-E2 (R441-L546) (Wang and Ha, 2004) and C. elegans APL1 (R327-Y434) (Hoopes et al., 2010).

### Sequence Alignment and Analysis of APP-E2 Conservation.

Sequences of APP, APLP1 and APLP2 were aligned using MUSCLE (Edgar, 2004) and manually edited to match the structural superposition of human copper bound APP-E2 and C. elegans APL1 (sequences are listed in supplementary data).

### GPC and SLS Measurements.

Analytical GPC and SLS studies were performed in 150 mM NaCl, 10 mM Sodium phosphate buffer, pH 7.3, on a calibrated (MWGF70, Sigma) Superdex200 10/300 GL column (GE Healthcare) using 50 μM APP-E2. Where applicable, a ˜3.6 kDa heparin preparation (Reviparin-Sodium, Abott) was added at 100 μM to the protein and the buffer. SLS data were recorded using VE 3580 RI and 270 Dual detectors (Viscotek, USA) and analyzed using software provided by the manufacturer. Given data represent average values±standard deviation of three independent experiments.

Metal Retention Assay.

50 μM APP-E2 (without His-tag) and 75 μM CuCl₂ in 50 mM Tris/HCl pH 7.3, 150 mM NaCl or 75 μM ZnCl₂ in 20 mM Hepes/NaOH pH 7.3, 150 mM NaCl was concentrated 8-fold at 20° C. using Vivaspin 500 ultrafiltration devices with 5.000 MWCO PES membranes (Sartorius stedim biotech). Samples without protein, or containing 50 μM lysozyme instead of APP-E2, served as controls. Metal ions in the retenate were quantified by a spectrophotometric method (Sabel et al., 2010). Each experiment was performed in triplicate and the average ratio of the change in concentration relative to the starting concentration (c-c₀/c₀) was plotted.

### Isothermal Titration Calorimetry (ITC).

ITC was performed at the same conditions as the metal retention assays in a NANO ITC calorimeter (TA Instruments, USA) adding 0.4 mM CuCl₂ in 6 μl injections or 0.4 mM ZnCl₂ in 11 μl injections to the cell containing 40 μM protein. Blank enthalphies for titrations of metals in buffer were subtracted from peak integrals. Binding constants were calculated with the NanoAnalyze program (TA Instruments, USA) using a model for a single set of identical binding sites in the case of copper binding. In the case of zinc binding the enthalpic data were fitted to a binding model representing two sets of independent binding sites, accounting for 1 strong binding site and 3.3 weak binding sites (4.3 total Zn²⁺ sites) per protein molecule (for further details see supplementary data). All titrations were performed in triplicate. The initially determined K_{Dapp} of the APP-E2/Cu complex had to be corrected for competition with the copper ligand Tris (Hatcher et al., 2008) and its pH-dependent protonation. The exact coordination chemistry of Cu²⁺ by Tris is, unfortunately, not precisely known, e.g. tertiary Cu(Tris)₂ and quinary Cu(Tris)₄ complexes are described in the literature. However, all models agree about the formation of a binary Tris-Cu²⁺ complex, described by a formation constant pK_{1(Tris)} of ˜4. The corrected K_{D} was calculated according to K_{D}=K_{Dapp}(1+ c_{Tris}×10^{pK}^{_{1(Tris)}}/(1+10^{-pH}×Kₐ))⁻¹ using pK_{1(Tris)}=3.82 (Canepari et al., 1999) and pK_{a(Tris)}=8.1.

### Limited Proteolysis.

Limited proteolysis was performed in 20 mM HEPES, 150 mM NaCl at pH 7.4 containing 0.5 μg/ml Trypsin (Calbiochem) and 20 μM purified protein (α-casein as control). Copper or zinc were supplemented at 40 μM to the Samples. Reactions were stopped after 30 and 60 mM by addition of 10 mM PMSF and analyzed by SDS-PAGE. Protease cleavage sites were characterized by Edman sequencing (Procise 494A, Applied Biosystems, Foster City, Calif., USA). Limited proteolysis results shown were reproduced in three independent experiments.

### Circular Dichroism (CD) Spectroscopy.

CD-data were collected on a J-710 spectropolarimeter (Jasco Corporation) in the same buffers used for ITC binding studies adding 7.5 μM APP-E2 and 10 μM zinc or copper. CD-signals were measured at 222 nm upon heating (1° C./min) of the samples from 20° C. to 90° C. The blank signals produced by the buffers at 20° C. were subtracted as constant values from the melting curves. Indicated melting points correspond to the inflection points of the melting curves determined from the peak of the 1st order derivative calculated using spectra analysis software (Jasco Corporation).

### APP-E2 Conservation Analysis.

The following Sequences were obtained from the UniProt (UP) database (UniProt, 2011) or GENEBANK (GB) to calculate the sequence alignments: Homo sapiens (H. s.) APP (UP:D3DSD1), Mus musculus (M. m.) APP (UP:Q6GR78), Gallus gallus (G. g.) APP (UP:Q9DGJ8), Xenopus laevis (X. l.) APP (UP:Q98SG0), Danio rerio (D. r.) APPa (UP:Q90W28), Drosophila melanogaster (D. m.) APPL (UP:P14599), Caenorhabditis elegans (C. e.) APL1 (GB:AAC46470.1), H. s. APLP1 (UP:P51693), H. s. APLP2 (UP: Q06481).

### ITC Measurements at High Zn/Protein Ratios.

The effect of excessive zinc to protein ratios was studied in ITC experiments in three repeating titrations into the same cell solution. Titration was performed in 20 mM Hepes/NaOH pH 7.3, 150 mM NaCl at 20° C., adding a 0.8 mM ZnCl₂ solution in 3×22 injections to 40 μM APP-E2, resulting in a final Zn²⁺/protein ratio of 22. The molar ratios for titration experiment 2 and 3 were calculated neglecting intermixture of cell volume and slight volume changes occurring after refill of the syringe. Blank enthalphies for titrations of metals in buffer were subtracted from peak integrals.

### Determination of Total Zn²⁺ Binding Sites on APP-E2.

The protein was saturated with 10 mM ZnCl₂ in 20 mM Hepes/NaOH pH 7.3, 150 mM NaCl resulting in quantitative precipitated of the protein. The precipitate was washed with buffer until a stable equilibrium ratio of zinc saturated protein in the liquid phase and the solid phase was reached. The concentration of Zn (Sabel et al., 2010) and protein (Bradford, 1976) were determined after each wash step in the liquid phase (supernatant) after 5 mM centrifugation at 16,000 g. The ratio between [Zn²⁺] and [APP-E2] at equilibrium conditions corresponds to the total number of zinc binding sites per protein molecule, which was determined to be 4.3+/−0.2 (FIG. 10B). Subtraction of one strong intra-molecular binding site yields the number of intermolecular binding sites of as 3.3.

### Biological Evaluation.

The following examplatory biological assays can be used to characterize the ability of agents of the invention to regulate the cleavage of APP, thereby reducing or inhibiting the production of amyloid beta.

### In Vitro Enzymatic BACE FRET (Fluorescence Resonance Energy Transfer) Assay.

The assay buffer is 0.05 M acetate, pH 4.2, 10% DMSO final, 100 μM genapol (which is a nonionic detergent, below it's Critical Micelle Concentration). Protease (0.2 nM) is pre-incubated for one hour with agents added in 1 μl of DMSO. Then the assay is started by the addition of FRET substrate (50 nM) and incubated for one hour. The FRET assay is terminated with by addition of Tris buffer, which raises the pH to neutrality, and the fluorescence is determined. The FRET substrate is a peptide with commercially available fluorophore and quencher, on opposite sides of the BACE cleavage site. Proteolytic cleavage of the FRET substrate releases quenching of fluorescence (excitation 488 nm and emission 425 nm).

Next to BACE-1, which has been shown to be the major β-secretase and BACE-2, any other protease can be employed including trypsin, elastase, thrombin, thermolysin, v8-endoproteinase, aminopeptidase, carboxypeptidase to name just a few. The agents has exhibit IC₅₀ values of 5 μM or less in the FRET in vitro enzyme assay are selected for further analysis.

### BACE Cell-Based Assay.

This cell-based assay measures inhibition or reduction of Abeta 40 in conditioned medium of agent treated cells expressing amyloid precursor protein. Cells stably expressing Amyloid Precursor Protein (APP) are plated at a density of 40K cells/well in 96 well plates (Costar). The cells are cultivated for 24 hours at 37° C. and 5% CO₂ in DMEM supplemented with 10% FBS. The agents are then added to cells in 10-point dose response concentrations with the starting concentration being either 100 μM or 10 μM. The agents are diluted from stock solutions in DMSO and the final DMSO concentration of the agents on cells is 0.1%. After 24 h of incubation with the agents the supernatant conditioned media is collected and the Abeta 40 levels are determined using a sandwich ELISA. The IC₅₀ of the agents is calculated from the percent of control or percent inhibition of Abeta 40 as a function of the concentration of the agents. The sandwich ELISA to detect Abeta 40 is performed in 96 well microtiter plates, which are pre-treated with goat anti-rabbit IgG (Pierce). The capture and detecting antibody pair that are used to detect Abeta 40 from cell supernatants are affinity purified pAb40 (Biosource) and biotinylated 6E10 (Signet Labs Inc.), respectively. The optimal concentration for the pAb40 antibody is 3 μg/ml in Superblock/TBS (Pierce) that is supplemented with 0.05% Tween 20 (Sigma). Optimal concentration for the detection antibody 6E10-biotinylated is 0.5 μg/ml in Superblock/TBS (Pierce) that is supplemented with 2% normal goat serum and 2% normal mouse serum. Cellular supernatants are incubated with the capture antibody for 3 h at 4° C., followed by 3 wash steps in TBS-tween (0.05%). The detecting antibody incubation was for 2 h at 4° C., again followed by the wash steps as described previously. The final readout of the ELISA is Time-Resolved Fluorescence (counts per minute) using Delfia reagents Streptavidin-Europium and Enhancement solutions (Perkin Elmer) and the Victor 2 multilabel counter (Perkin Elmer). Agents that exhibit activities with IC₅₀ values of 5 μM or less in the cell-based assay are further selected for in vivo testing.

It will be appreciated that the methods and agents of the instant invention can be incorporated in the form of a variety of embodiments, only a few of which are disclosed herein. It will be apparent to the person skilled in the art that other embodiments exist and do not depart from the spirit of the invention. Thus, the described embodiments are illustrative and should not be construed as restrictive.

TABLE I
Data collection and refinement statistics

| | | | | | | |
|---|---|---|---|---|---|---|
| Dataset | Cd²⁺ lowE | Cd²⁺ | Zn²⁺ Peak | Zn ⁺ lowE | Cu²⁺ Peak | Cu²⁺ lowE |

Data collection statistics

| Wavelength (Å) | 1,600 | 0.918 | 1.282 | 1.284 | 1.377 | 1.382 |
|---|---|---|---|---|---|---|
| Resolution limit(Å) | 2.7 | 2.0 | 2.4 | 2.5 | 2.6 | 2.7 |
| Unit cell parameters | P4₃ | | | | | |
| a (Å) | 39.3 | 39.7 | 39.7 | | 39.8 | |
| c (Å) | 125.9 | 126.2 | 125.8 | | 126.5 | |
| Completeness (%)^{b} | 100.0 (100.0) | 100.0 (100.0) | 98.3 (89.0) | 99.7 (98.4) | 98.2 (88.3) | 99.8 (99.4) |
| Rmerge (%)^{b} | 9.1 (42.2) | 5.6 (38.6) | 2.9 (7.9) | 3.4 (7.9) | 3.4 (9.9) | 3.6 (10.2) |
| <I/σ_{I}>^{b} | 46.2 (8.7) | 14.0 (3.4) | 27.1 (12.1) | 22.8 (13.2) | 23.5 (10.0) | 21.1 (10.1) |
| Redundancy ^{b} | 43.2 (33.8) | 4.2 (4.1) | 4.0 (3.2) | 4.1 (4.0) | 4.1 (3.5) | 4.0 (3.8) |

Refinement statistics

| | | | |
|---|---|---|---|
| Structure | Cd²⁺ | Zn²⁺ | Cu²⁺ |
| Resolution (Å) | 28.0-2.0 | 28.0-2.4 | 25.7-2.6 |
| Unique Reflections^{a} | 12,503/647 | 7,020/433 | 5597/341 |
| R_{work} (%)/R_{free} (%) | 19.8/23.7 | 20.4/24.5 | 20.6/23.9 |
| Nonhydrogen atoms: | | | |
| Protein | 1,509 | 1,535 | 1,543 |
| Water | 95 | 79 | 63 |
| Other | 17 | 13 | 13 |
| B-Factors: | | | |
| Overall/Wilson plot (Å²) | 41.0/31.2 | 36.7/40.2 | 39.7/50.0 |
| rms deviations: | | | |
| Bond lengths (Å) | 0.0073 | 0.0049 | 0.0048 |
| Bonded B-factors (Å²) | 3.6 | 3.1 | 2.6 |

^{a}work/free
^{b}Values given in parenthesis represent the highest resolution shell. (Data collection: Cd²⁺ lowE: 2.85-2.70 Å; Cd²⁺: 2.11-2.00 Å; Zn²⁺ Peak: 2.53-2.40 Å; Zn²⁺ lowE: 2.64-2.50 Å; Cu²⁺ Peak: 2.74-2.60 Å; Cu²⁺ lowE: 2.85-2.70 Å Refinement: Cd²⁺: 2.09-2.00 Å; Zn²⁺: 2.40-2.51 Å; Cu²⁺: 2.60-2.72 Å)

TABLE LL
Heparin-induced changes in Molecular weight

| | MWᵣₕ (kDa) | MW_{SLS} (kDa) |
|---|---|---|
| APP-E2 − HP | 27.4 ± 0.2 | 27.0 ± 0.3 |
| APP-E2 + HP | 39.2 ± 0.4 | 31.6 ± 0.2 |

TABLE III
Binding constants of Cu²⁺ and Zn to APP-E2 determined by ITC

| Cu-APP-E2 | | |
|---|---|---|
| K_{Dapp} | K_{D} | Zn-APP-E2 |

| | | | |
|---|---|---|---|
| K_{D1} (μM) | 0.61 ± 0.23 | 0.013 ± 0.005 | 3.9 ± 1.5 |
| K_{D2} (mM) | — | | 2.6 ± 1.2^{a} |
| dH₁ (kJ/mol) | −19.9 ± 0.9 | | −16.6 ± 0.6 |
| dH₂ (kJ/mol) | — | | 1200 ± 600 |
| n₁ | 0.70 ± 0.03 | | 0.7 ± 0.2 |
| n₂ | — | | 3.3^{b} |

^{a}Not statistically significant in curve fit as confidence intervals exceeds the measured values at a confidence level of 99%.
^{b}Determined independently and fixed during curve fit.

TABLE LV
Fragments generated by limited trypsin digestion

| | | | Influence on |
|---|---|---|---|
| Fragment^{a} | N-terminal sequence | Cleavage site | cleavage site |
| 1^{b} | STPD | | |
| 2 | AVID | K354↓A355 | Cu²⁺ |
| 3 | QQLV | R375↓Q376 | Cu²⁺ |
| 4 | VEAM | R385↓V386 | Zn²⁺; Cu²⁺ |

^{a}Numbers according to FIG. 6.
^{b}Cleavage within C-terminal residues, not contained in the native APP-sequence, but remaining after Tag-cleavage with V8.

TABLE V
Interaction within the N-terminal coiled coil of different E2-structures

| | Residues | | | Free energy |
|---|---|---|---|---|
| Molecule/ | in | Residues in | Interaction | of interaction |
| structure^{a} | helix α-B | helix α-C | interface [Å2]^{b} | ΔiG [kcal/mol]^{b} |
| this work | 313-347 | 350-388 | 816 | −13.7 |
| 1RW6 | 369-403 | 406-444 | 771 | −18.5 |
| 3K66 | 248-282 | 292-330 | 754 | −17.2 |

^{a}The interaction interface per protomer and ΔiG, the solvation free energy gain upon interface formation (corresponds to hydrophobic interactions) was calculated with PISA.
^{b}Pdb-id: 1RW6 {Wang, 2004 #79}, APP-E2; Pdb-id: 3K66 {Hoopes, 2010 #43}, APL-1

### BIBLIOGRAPHY

Acevedo, K. M., Hung, Y. H., Dalziel, A. H., Li, Q. X., Laughton, K., Wikhe, K., Rembach, A., Roberts, B., Masters, C. L., Bush, A. I. and Camakaris, J. (2011) Copper promotes the trafficking of the amyloid precursor protein. J Biol Chem, 286, 8252-8262.
Allen, F. H. (2002) The Cambridge Structural Database: a quarter of a million crystal structures and rising. Acta Crystallogr B, 58, 380-388.
Andersen, O. M., Schmidt, V., Spoelgen, R., Gliemann, J., Behlke, J., Galatis, D., McKinstry,
W. J., Parker, M. W., Masters, C. L., Hyman, B. T., Cappai, R. and Willnow, T. E. (2006) Molecular dissection of the interaction between amyloid precursor protein and its neuronal trafficking receptor SorLA/LR11. Biochemistry, 45, 2618-2628.
Anliker, B. and Muller, U. (2006) The functions of mammalian amyloid precursor protein and related amyloid precursor-like proteins. Neurodegener Dis, 3, 239-246.
Banci, L., Bertini, I., Boca, M., Calderone, V., Cantini, F., Girotto, S, and Vieru, M. (2009) Structural and dynamic aspects related to oligomerization of apo SOD1 and its mutants. Proc Nall Acad Sci USA, 106, 6980-6985.
Barker, W. W., Luis, C. A., Kashuba, A., Luis, M., Harwood, D. G., Loewenstein, D., Waters, C., Jimison, P., Shepherd, E., Sevush, S., Graff-Radford, N., Newland, D., Todd, M., Miller, B., Gold, M., Heilman, K., Doty, L., Goodman, I., Robinson, B., Pearl, G., Dickson, D. and Duara, R. (2002) Relative frequencies of Alzheimer disease, Lewy body, vascular and frontotemporal dementia, and hippocampal sclerosis in the State of Florida Brain Bank. Alzheimer Dis Assoc Disord, 16, 203-212.
Bayer, T. A. and Multhaup, G. (2006) The Role of Metal Ions in the Amyloid Precursor Protein and in Alzheimer's Disease. In Sigel, A., Sigel, H. and Sigel, R. K. O. (eds.), Neurodegenerative Diseases and Metal Ions. John Wiley & Sons, Ltd, Chichester, UK, Vol. 1
Blennow, K., de Leon, M. J. and Zetterberg, H. (2006) Alzheimer's disease. Lancet, 368, 387-403.
Bode, W., Gomis-Ruth, F. X. and Stockier, W. (1993) Astacins, serralysins, snake venom and matrix metalloproteinases exhibit identical zinc-binding environments (HEXXHXXGXXH and Met-turn) and topologies and should be grouped into a common family, the ‘metzincins’. FEBS Lett, 331, 134-140.
Boll, M. C., Alcaraz-Zubeldia, M., Montes, S, and Rios, C. (2008) Free copper, ferroxidase and SOD1 activities, lipid peroxidation and NO(x) content in the CSF. A different marker profile in four neurodegenerative diseases. Neurochem Res, 33, 1717-1723.
Bradford, M. M. (1976) A rapid and sensitive method for the quantitation of microgram quantities of protein utilizing the principle of protein-dye binding. Anal Biochem, 72, 248-254.
Brunger, A. T. (2007) Version 1.2 of the Crystallography and NMR system. Nat Protoc, 2, 2728-2733.
Bush, A. I., Multhaup, G., Moir, R. D., Williamson, T. G., Small, D. H., Rumble, B., Pollwein, P., Beyreuther, K. and Masters, C. L. (1993) A novel zinc(II) binding site modulates the function of the beta A4 amyloid protein precursor of Alzheimer's disease. J Biol Chem, 268, 16109-16112.
Canepari, S., Carunchio, V., Castellano, P. and Messina, A. (1999) Complex formation equilibria of some beta-amino-alcohols with lead(II) and cadmium(II) in aqueous solution. Talanta, 47, 1077-1084.
Cappai, R., Cheng, F., Ciccotosto, G. D., Needham, B. E., Masters, C. L., Multhaup, G., Fransson, L. A. and Mani, K. (2005) The amyloid precursor protein (APP) of Alzheimer disease and its paralog, APLP2, modulate the Cu/Zn-Nitric Oxide-catalyzed degradation of glypican-1 heparan sulfate in vivo. J Biol Chem, 280, 13913-13920.
Clement, A. B., Gimpl, G. and Behl, C. (2010) Oxidative stress resistance in hippocampal cells is associated with altered membrane fluidity and enhanced nonamyloidogenic cleavage of endogenous amyloid precursor protein. Free Radic Biol Med, 48, 1236-1241.
Cowtan, K. (2006) The Buccaneer software for automated model building. 1. Tracing protein chains. Acta Crystallogr D Biol Crystallogr, 62, 1002-1011.
Dahms, S. O., Hoefgen, S., Roeser, D., Schlott, B., Guhrs, K. H. and Than, M. E. (2010) Structure and biochemical analysis of the heparin-induced E1 dimer of the amyloid precursor protein. Proc Natl Acad Sci USA, 107, 5381-5386.
de La Fortelle, E. and Bricogne, G. (1997) [27] Maximum-likelihood heavy-atom parameter refinement for multiple isomorphous replacement and multiwavelength anomalous diffraction methods. In Charles W. Carter, Jr. (ed.), Methods in Enzymology. Academic Press, Vol. Volume 276, pp. 472-494.
Duce, J. A. and Bush, A. I. (2010) Biological metals and Alzheimer's disease: implications for therapeutics and diagnostics. Prog Neurobiol, 92, 1-18.
Duce, J. A., Tsatsanis, A., Cater, M. A., James, S. A., Robb, E., Wikhe, K., Leong, S. L., Perez, K., Johanssen, T., Greenough, M. A., Cho, H. H., Galatis, D., Moir, R. D., Masters, C. L., McLean, C., Tanzi, R. E., Cappai, R., Barnham, K. J., Ciccotosto, G. D., Rogers, J. T. and Bush, A. I. (2010) Iron-export ferroxidase activity of beta-amyloid precursor protein is inhibited by zinc in Alzheimer's disease. Cell, 142, 857-867.
Dulubova, I., Ho, A., Huryeva, I., Sudhof, T. C. and Rizo, J. (2004) Three-dimensional structure of an independently folded extracellular domain of human amyloid-beta precursor protein. Biochemistry, 43, 9583-9588.
Edgar, R. C. (2004) MUSCLE: multiple sequence alignment with high accuracy and high throughput. Nucleic Acids Res, 32, 1792-1797.
Frederickson, C. J., Giblin, L. J., 3rd, Balaji, R. V., Masalha, R., Frederickson, C. J., Zeng, Y., Lopez, E. V., Koh, J. Y., Chorin, U., Besser, L., Hershfinkel, M., Li, Y., Thompson, R. B. and Krezel, A. (2006) Synaptic release of zinc from brain slices: factors governing release, imaging, and accurate calculation of concentration. J Neurosci Methods, 154, 19-29.
Gifford, J. L., Walsh, M. P. and Vogel, H. J. (2007) Structures and metal-ion-binding properties of the Ca2+-binding helix-loop-helix EF-hand motifs. Biochem J, 405, 199-221.
Goodman, Y. and Mattson, M. P. (1994) Secreted forms of beta-amyloid precursor protein protect hippocampal neurons against amyloid beta-peptide-induced oxidative injury. Exp Neurol, 128, 1-12.
Gralle, M. and Ferreira, S. T. (2007) Structure and functions of the human amyloid precursor protein: the whole is more than the sum of its parts. Prog Neurobiol, 82, 11-32.
Gralle, M., Oliveira, C. L., Guerreiro, L. H., McKinstry, W. J., Galatis, D., Masters, C. L., Cappai, R., Parker, M. W., Ramos, C. H., Torriani, I. and Ferreira, S. T. (2006) Solution conformation and heparin-induced dimerization of the full-length extracellular domain of the human amyloid precursor protein. J Mol Biol, 357, 493-508.
Groemer, T. W., Thiel, C. S., Holt, M., Riedel, D., Hua, Y., Huve, J., Wilhelm, B. G. and Klingauf, J. (2011) Amyloid Precursor Protein Is Trafficked and Secreted via Synaptic Vesicles. PLoS One, 6, e18754.
Grum, V. L., Li, D., MacDonald, R. I. and Mondragon, A. (1999) Structures of two repeats of spectrin suggest models of flexibility. Cell, 98, 523-535.
Haass, C. and Selkoe, D. J. (2007) Soluble protein oligomers in neurodegeneration: lessons from the Alzheimer's amyloid beta-peptide. Nat Rev Mol Cell Biol, 8, 101-112.
Han, Q., Fu, Y., Zhou, H., He, Y. and Luo, Y. (2007) Contributions of Zn(II)-binding to the structural stability of endostatin. FEBS Lett, 581, 3027-3032.
Hatcher, L. Q., Hong, L., Bush, W. D., Carducci, T. and Simon, J. D. (2008) Quantification of the binding constant of copper(II) to the amyloid-beta peptide. J Phys Chem B, 112, 8160-8164.
Hesse, L., Beher, D., Masters, C. L. and Multhaup, G. (1994) The beta A4 amyloid precursor protein binding to copper. FEBS Lett, 349, 109-116.
Ho, A. and Sudhof, T. C. (2004) Binding of F-spondin to amyloid-beta precursor protein: a candidate amyloid-beta precursor protein ligand that modulates amyloid-beta precursor protein cleavage. Proc Natl Acad Sci USA, 101, 2548-2553.
Hoe, H. S., Wessner, D., Beffert, U., Becker, A. G., Matsuoka, Y. and Rebeck, G. W. (2005) F-spondin interaction with the apolipoprotein E receptor ApoEr2 affects processing of amyloid precursor protein. Mol Cell Biol, 25, 9259-9268.
Hoey, S. E., Williams, R. J. and Perkinton, M. S. (2009) Synaptic NMDA receptor activation stimulates alpha-secretase amyloid precursor protein processing and inhibits amyloid-beta production. J Neurosci, 29, 4442-4460.
Hoopes, J. T., Liu, X., Xu, X., Demeler, B., Folta-Stogniew, E., Li, C. and Ha, Y. (2010) Structural characterization of the E2 domain of APL-1, a Caenorhabditis elegans homolog of human amyloid precursor protein, and its heparin binding site. J Biol Chem, 285, 2165-2173.
Hung, Y. H., Bush, A. I. and Chemy, R. A. (2010) Copper in the brain and Alzheimer's disease. J Biol Inorg Chem, 15, 61-76.
Hung, Y. H., Robb, E. L., Volitakis, I., Ho, M., Evin, G., Li, Q. X., Culvenor, J. G., Masters, C. L., Chemy, R. A. and Bush, A. I. (2009) Paradoxical condensation of copper with elevated beta-amyloid in lipid rafts under cellular copper deficiency conditions: implications for Alzheimer disease. J Biol Chem, 284, 21899-21907.
Kaden, D., Munter, L. M., Joshi, M., Treiber, C., Weise, C., Bethge, T., Voigt, P., Schaefer, M., Beyermann, M., Reif, B. and Multhaup, G. (2008) Homophilic interactions of the amyloid precursor protein (APP) ectodomain are regulated by the loop region and affect beta-secretase cleavage of APP. J Biol Chem, 283, 7271-7279.
Kaden, D., Voigt, P., Munter, L. M., Bobowski, K. D., Schaefer, M. and Multhaup, G. (2009) Subcellular localization and dimerization of APLP1 are strikingly different from APP and APLP2. J Cell Sci, 122, 368-377.
Keil, C., Huber, R., Bode, W. and Than, M. E. (2004) Cloning, expression, crystallization and initial crystallographic analysis of the C-terminal domain of the amyloid precursor protein APP. Acta Crystallogr D Biol Crystallogr, 60, 1614-1617.
Kong, G. K., Adams, J. J., Harris, H. H., Boas, J. F., Curtain, C. C., Galatis, D., Masters, C. L., Barnham, K. J., McKinstry, W. J., Cappai, R. and Parker, M. W. (2007) Structural studies of the Alzheimer's amyloid precursor protein copper-binding domain reveal how it binds copper ions. J Mol Biol, 367, 148-161.
Krissinel, E. and Henrick, K. (2007) Inference of macromolecular assemblies from crystalline state. J Mol Biol, 372, 774-797.
Lee, S., Xue, Y., Hu, J., Wang, Y., Liu, X., Demeler, B. and Ha, Y. (2011) The E2 domains of APP and APLP1 share a conserved mode of dimerization. Biochemistry.
Lezoualc'h, F., Engert, S., Berning, B. and Behl, C. (2000) Corticotropin-releasing hormone-mediated neuroprotection against oxidative stress is associated with the increased release of non-amyloidogenic amyloid beta precursor protein and with the suppression of nuclear factor-kappaB. Mol Endocrinol, 14, 147-159.
Lichtenthaler, S. F., Haass, C. and Steiner, H. (2011) Regulated intramembrane proteolysis-lessons from amyloid precursor protein processing. J. Neurochem.
Liljas, A., Kannan, K. K., Bergsten, P. C., Waara, I., Fridborg, K., Strandberg, B., Carlbom, U., Jarup, L., Lovgren, S, and Petef, M. (1972) Crystal structure of human carbonic anhydrase C. Nat New Biol, 235, 131-137.
McCoy, A. J., Grosse-Kunstleve, R. W., Adams, P. D., Winn, M. D., Storoni, L. C. and Read, R. J. (2007) Phaser crystallographic software. J Appl Crystallogr, 40, 658-674.
Multhaup, G., Bush, A. I., Pollwein, P. and Masters, C. L. (1994) Interaction between the zinc (II) and the heparin binding site of the Alzheimer's disease beta A4 amyloid precursor protein (APP). FEBS Lett, 355, 151-154.
Paoletti, P., Vergnano, A. M., Barbour, B. and Casado, M. (2009) Zinc at glutamatergic synapses. Neuroscience, 158, 126-136.
Parsons, M. R., Convery, M. A., Wilmot, C. M., Yadav, K. D., Blakeley, V., Corner, A. S., Phillips, S. E., McPherson, M. J. and Knowles, P. F. (1995) Crystal structure of a quinoenzyme: copper amine oxidase of Escherichia coli at 2 Å resolution. Structure, 3, 1171-1184.
Project, C. C. (1994) The CCP4 suite: programs for protein crystallography. Acta Crystallogr D Biol Crystallogr, 50, 760-763.
Ramachandran, G. N. and Sasisekharan, V. (1968) Conformation of polypeptides and proteins. Adv Protein Chem, 23, 283-438.
Reinhard, C., Hebert, S. S, and De Strooper, B. (2005) The amyloid-beta precursor protein: integrating structure with biological function. Embo J, 24, 3996-4006.
Sabel, C. E., Neureuther, J. M. and Siemann, S. (2010) A spectrophotometric method for the determination of zinc, copper, and cobalt ions in metalloproteins using Zincon. Anal Biochem, 397, 218-226.
Sabo, S. L., Ikin, A. F., Buxbaum, J. D. and Greengard, P. (2003) The amyloid precursor protein and its regulatory protein, FE65, in growth cones and synapses in vitro and in vivo. J Neurosci, 23, 5407-5415.
Scheuermann, S., Hambsch, B., Hesse, L., Stumm, J., Schmidt, C., Beher, D., Bayer, T. A., Beyreuther, K. and Multhaup, G. (2001) Homodimerization of amyloid precursor protein and its implication in the amyloidogenic pathway of Alzheimer's disease. J Biol Chem, 276, 33923-33929.
Schubert, W., Prior, R., Weidemann, A., Dircksen, H., Multhaup, G., Masters, C. L. and Beyreuther, K. (1991) Localization of Alzheimer beta A4 amyloid precursor protein at central and peripheral synaptic sites. Brain Res, 563, 184-194.
Soba, P., Eggert, S., Wagner, K., Zentgraf, H., Siehl, K., Kreger, S., Lower, A., Langer, A., Merdes, G., Paro, R., Masters, C. L., Muller, U., Kins, S, and Beyreuther, K. (2005) Homo- and heterodimerization of APP family members promotes intercellular adhesion. Embo J, 24, 3624-3634.
Stein, N. (2008) CHAINSAW: a program for mutating pdb files used as templates in molecular replacement. J Appl Crystallogr, 41, 641-643.
Tainer, J. A., Getzoff, E. D., Richardson, J. S, and Richardson, D. C. (1983) Structure and mechanism of copper, zinc superoxide dismutase. Nature, 306, 284-287.
Than, M. E., Henrich, S., Bourenkov, G. P., Bartunik, H. D., Huber, R. and Bode, W. (2005) The endoproteinase furin contains two essential Ca2+ ions stabilizing its N-terminus and the unique 51 specificity pocket. Acta Crystallogr D Biol Crystallogr, 61, 505-512.
Turk, D. (1992) Weiterentwicklung eines Programms für Molekülgraphik und Elektronendichte-Manipulation und seine Anwendung auf verschiedene Protein Strukturaufklärungen. PhD thesis (Technische Universität München, Munich, Germany).
The UniProt Consortium (2011) Ongoing and future developments at the Universal Protein Resource. Nucleic Acids Res, 39, D214-219.
Wang, Y. and Ha, Y. (2004) The X-ray structure of an antiparallel dimer of the human amyloid precursor protein E2 domain. Mol Cell, 15, 343-353.
Watt, N. T., Whitehouse, I. J. and Hooper, N. M. (2010) The role of zinc in Alzheimer's disease. Int J Alzheimers Dis, 2011, 971021.
Wimo, A., Winblad, B. and Jonsson, L. (2010) The worldwide societal costs of dementia: Estimates for 2009. Alzheimers Dement, 6, 98-103.

1. A method for selecting an agent that influences the conformation of an isolated amyloid precursor protein (APP) E2 core domain comprising
providing a sample comprising said APP-E2 core domain having a first conformation, adding a candidate agent to said sample to induce either an alteration or no alteration in said first conformation,
determining whether said candidate agent induces said alteration in said first conformation to result in a second conformation of said APP-E2 core domain, and selecting said agent, wherein said agent is the candidate agent that induces said alteration of said first conformation to result in said second conformation.

2. The method of claim 1, wherein said first conformation is an unrestricted conformation and said second conformation is a restricted conformation.

3. The method of claim 1, wherein said first conformation is a restricted conformation and said second conformation is an unrestricted conformation.

4. The method of claim 1, wherein said first conformation is a first restricted conformation and said second conformation is a second restricted conformation.

5. The method of claim 1, wherein said APP-E2 core domain is part of a APP-E2 domain or a full length APP molecule or a fragment thereof comprising the APP-E2 core domain.

6. The method of claim 1, wherein the alpha helical content of said APP-E2 core domain in said first conformation is at least 5%, 10%, 20%, 30% or 40% higher or lower than the alpha helical content of said APP-E2 core domain in said second conformation.

7. The method of claim 1, wherein whether said candidate agent induces said alteration in said first conformation is determined by measuring the melting temperature of said APP-E2 core domain subsequent to addition of said candidate agent and comparing said melting temperature to the melting temperature of the APP-E2 core domain prior to addition of said candidate agent or melting temperatures provided for another APP-E2 core domain.

8. The method of claim 7, wherein upon determining a melting temperature difference between the first and second conformation of more than 1, 2, 3, 4, 5, 6, 7, 8, 9, 10° C., said candidate agent is selected as an agent.

9. The method of claim 1, wherein whether said candidate agent induces said alteration in said first conformation is determined by subjecting said APP-E2 core domain subsequent to addition of said candidate agent to a spectroscopic method to obtain a spectrogram or nuclear magnetic resonance (NMR) to obtain a NMR image and comparing the spectrogram or NMR image with the spectrogram or NMR image of the respective APP-E2 core domain prior to addition of said candidate agent or an NMR image provided for another APP-E2 core domain.

10. The method of claim 1, wherein whether said candidate agent induces said alteration in said first conformation is determined by digesting said APP-E2 core domain subsequent to addition of said candidate agent with a protease and comparing the digestion pattern with a digesting pattern of the respective APP-E2 core domain prior to addition of said candidate agent or digestion patterns provided for another APP-E2 core domain.

11. The method of claim 2, wherein the APP-E2 core domain comprises an alpha B helix and an alpha C helix and wherein, in said restricted conformation, an N-terminal part of the alpha C helix and the alpha B helix are rotated by or by more than 5°, 6°, 7°, 8°, 9°, 10°, 11° or 12° around a hinge region in the alpha C helix towards an N-terminal end of the alpha D helix.

12. The method of claim 11, wherein said rotation occurs around a hinge region in the alpha C helix.

13. The method of claim 12, wherein the hinge region is located C terminally of a histidine residue in the alpha C helix that is part of the metal ion binding site M1.

14. The method of claim 11, wherein in said second conformation the alpha B helix and at said alpha C helix are shifted out of alignment to each other by at least 5° or remain parallel to each other.

15. The method of claim 1, wherein a transition from said first conformation to said second conformation or from a second to a first conformation correlates to a more than 10%, 20%, 30%, 40% or 50% change of protease cleavage at an arginine in the alpha C helix of the APP-E2 domain.

16. The method of claim 2, wherein said sample comprising said APP-E2 core domain in a unrestricted conformation is metal free.

17. The method of claims 3, wherein prior to adding the candidate agent to said sample a transition metal ion is added to said APP-E2 core protein to provide said restricted conformation.

18. The method of claim 1, wherein said sample is an in vitro sample comprising said APP-E2 core domain attached to a sensor chip or said APP-E2 core domain in solution.

19. The method of claim 1, wherein said APP-E2 core domain comprises amino acids corresponding to amino acids 301, 303, 312, 313, 334, 338, 349, 360, 361, 367, 368, 382, 385, 386, 388, 391, 395, 397, 404, 405, 410, 419, 424, 425, 428, 432, 439, 448, 459, 462, 467, 473, 477 amino acids of the human APP695 protein.

20. The method of claim 1, wherein the agent interacts with histidine residues corresponding to histidine residues 382, 432 and 436 of human APP 695.

21. The method of claim 1 further comprising
adding the agent selected to an APP molecule,
subjecting said APP molecule to a protease, and
evaluating the digestion products.

22. The method of claim 21, wherein the digestion product is the product of a limited proteolysis by trypsin, elastase, thrombin, thermolysin, v8-endoproteinase, aminopeptidase or carboxypeptidase.

23. The method of claim 21, wherein said digestion product is obtained via an in vitro alpha secretase and/or beta secretase assay.

24. The method of claim 23, wherein the presence of one or more Abeta peptide species are detected by one or more antibodies that detect said one or more Abeta peptide species.

25. An agent selected via the method of claim 1.

26. The agent of claim 25, wherein the agent binds histidine residues corresponding to histidine residues 382, 432 and 436 of the human APP695.

27. A method for designing, selecting and/or optimizing a chemical entity that binds to all or part of an APP-E2 core domain comprising:
(a) employing the structural coordinates of the APP-E2 core domain according to any one of FIGS. 1-3 and/or 6 to generate a three-dimensional model of said APP-E2 core domain comprising metal binding site M1 on a computer, wherein said computer comprises the tools for generating said three-dimensional model;
(b) identifying in said model at least three histidines forming a metal binding site M1, wherein the histidines are located in an alpha B, alpha C and alpha D helix of the APP-E2 core domain,
(c) employing the structural coordinates in (a) for designing, selecting and/or optimizing, said chemical entity by performing a conformation shifting operation between said chemical entity which shifts a first conformation of said APP-E2 core domain to a second conformation, wherein in said second conformation:
(i) a distance of at least two of the three histidines widens or narrows by least 2 Å,
(ii) a distance of the alpha C and alpha D helix widens or narrows by least 2 Å, and/or
(iii) an N terminal part of the alpha C helix and the alpha B helix are rotated by or by more than 5°, 6°, 7°, 8°, 9°, 10°, 11° or 12° around a hinge region in the alpha C helix towards an N-terminal end of the alpha D helix.

28. The method of claim 27, wherein said chemical entity is a candidate agent or an agent.

29. The method of claim 27, wherein in (iii), the alpha C helix and the alpha B helix have parallel alignment or are shifted out of alignment during said rotation by at least 5°.


SEQUENCE LISTING (18 sequences)

SEQ ID NO: 1 - protein, 206 aa, Homo sapiens
STPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDD

SEQ ID NO: 2 - protein, 206 aa, Mus musculus
STPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDD

SEQ ID NO: 3 - protein, 206 aa, Gallus gallus
STPDAVDKYLETPGDENEHAHFQKAKERLEAKHRERMSQVMREWEEAERQAKNLPKADKKAVIQHFQEKVESLEQEAANERQQLVETHMARVEAMLNDRRRIALENYITALQTVPPRPRHVFNMLKKYVRAEQKDRQHTLKHFEHVRMVDPKKAAQIRSQVMTHLRVIYERMNQSLSFLYNVPAVAEEIQDEVDELLQKEQNYSDD

SEQ ID NO: 4 - protein, 206 aa, Xenopus laevis
STPDAVDKYLENPNDENEHDRFLKAKERLEGKHREKMSEVMKEWEEAERQAKNLPKADKKAVIQHFQEKVESLEQEAANERQQLVETHMARVEAMLNDRRRIALENYITALQADPPRPRHVFNMLKKYVRAEQKDRQHTLKHFEHVRMVDPKKAAQIRSQVMTHLRVINERMNQSFSLLYKVPAVAEEIQDEVDELFQKEQNYSDD

SEQ ID NO: 5 - protein, 200 aa, Danio rerio
SPPDAVDRYLETPADENEHAHFLKAKESLETKHRERMSQVMREWEEAERQAKSLPRNDKKAVIQHFQEKVEALEQESASERQQLVETHMARVEALLNDRRRLALESYLSALQADPPRPRHVFSLLKKYVRAEQKDRQHTLKHFEHVRMVDPKKAAQIRPQVLTHLRVIEERMNQSLGLLYKVPGVADDIQDQVELLQREQ

SEQ ID NO: 6 - protein, 203 aa, Drosophila melanogaster
DPYFTHFDPHYEHQSYKVSQKRLEESHREKVTRVMKDWSDLEEKYQDMRLADPKAAQSFKQRMTARFQTSVQALEEEGNAEKHQLAAMHQQRVLAHINQRKREAMTCYTQALTEQPPNAHHVEKCLQKLLRALHKDRAHALAHYRHLLNSGGPGGLEAAASERPRTLERLIDIDRAVNQSMTMLKRYPELSAKIAQLMNDYIL

SEQ ID NO: 7 - protein, 211 aa, Caenorhabditis elegans
SQDPYFKIANWTNEHDDFKKAEMRMDEKHRKKVDKVMKEWGDLETRYNEQKAKDPKGAEKFKSQMNARFQKTVSSLEEEHKRMRKEIEAVHEERVQAMLNEKKRDATHDYRQALATHVNKPNKHSVLQSLKAYIRAEEKDRMHTLNRYRHLLKADSKEAAAYKPTVIHRLRYIDLRINGTLAMLRDFPDLEKYVRPIAVTYWKDYRDEVSP

SEQ ID NO: 8 - protein, 197 aa, Homo sapiens
DGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSE

SEQ ID NO: 9 - protein, 199 aa, Homo sapiens
PTNDVDVYFETSADDNEHARFQKAKEQLEIRHRNRMDRVKKEWEEAELQAKNLPKAERQTLIQHFQAMVKALEKEAASEKQQLVETHLARVEAMLNDRRRMALENYLAALQSDPPRPHRILQALRRYVRAENKDRLHTIRHYQHVLAVDPEKAAQMKSQVMTHLHVIEERRNQSLSLLYKVPYVAQEIQEEIDELLQEQ

SEQ ID NO: 10 - protein, 695 aa, Homo Sapiens
  MISC_FEATURE: APP695
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 11 - protein, 695 aa, Mus musculus
  MISC_FEATURE: APP
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 12 - protein, 695 aa, Gallus gallus
  MISC_FEATURE: APP
MLPHLALLLLAAGAARALEVPADGNAGLLAEPQIAMFCGKLNMHMNVQNGKWESDPSGTKTCIDTKEGILQYCQEVYPELQITNVVEANQPVTIQNWCKRGWKQCNGHPHIVVPYRCLVGEFVSDALLVPDKCKLLHQERMDVCETHLHWHTVAKESCSEKSMNLHDYGMLLPCGIDKFRGVEFVCCPLAEESDNLDSADAEDDDSDVWWGGADADYADGSDDKVVEEQPEEDEELTVVEDEDADDDDDDDGDEIEETEEEYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRIALENYITALQTVPPRPRHVFNMLKKYVRAEQKDRQHTLKHFEHVRMVDPKKAAQIRSQVMTHLRVIYERMNQSLSFLYNVPAVAEEIQDEVDELLQKEQNYSDDVLANMISEPRISYGNDALMPSLTETKTTVELLPVDGEFSLDDLQPWHPFGVDSVPANTENEVEPVDARPAADRGLTTRPGSGLTNVKTEEVSEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 13 - protein, 693 aa, Xenopus laevis
  MISC_FEATURE: APP
MLPHITLLVLTVGALALEVPADGNGGLLAEPQIAMFCGKLNMHMNVQNGKWETDVSGTKGCIGTKEGILQYCQEVYPELQITNVVEANQPVTIQNWCKKGRKQCKSRTHIVVPYRCLVGEFVSDALLVPDKCKFLHQERMDICETHLHWHTVAKESCSEKSMSLHEYGMLLPCGIDKFRGVEFVCCPSAEESESFDSADAEDDSDAWWGGADADYVDRSDDKAVEAQPDEEEEVVEVEEEETDDDEDDGDEAEEEPEEPYEEATERTTSIATTTTTTTESVEEVVRVPATAASTPDAVDKYLENPNDENEHDRFLKAKERLEGKHREKMSEVMKEWEEAERQAKNLPKADKKAVIQHFQEKVESLEQEAANERQQLVETHMARVEAMLNDRRRIALENYITALQADPPRPRHVFNMLKKYVRAEQKDRQHTLKHFEHVRMVDPKKAAQIRSQVMTHLRVINERMNQSFSLLYKVPAVAEEIQDEVDELFQKEQNYSDDMVSNMVSDHRVSYGNDALMPSLSETKTTVELLPVDGEFNIEDLQPWHSFGVDSVPANTENEVEPVDARPAADRGLTTRPGSGLTNIKTEEISEVKMDSEYRHDTAYEVHHQKLVFFAEEVGSNKGAIIGLMVGGVVIATVIVITLVMLKKKQYTTIHHGVVEVDAAVTPEERHLTKMQQNGYENPTYKFFEQMQN

SEQ ID NO: 14 - protein, 738 aa, Danio rerio
  MISC_FEATURE: APPa
MRSRELFILLMAVASTLAVEVPSDSGTGLLAEPQIAMFCGKLNMHINIQSGKWEPDPSGSKSCIGNKEGILQYCQEVYPELQITNVVEANQPVSIWDWCKKSRKQCRSHMHIVVPYRCLVGEFVSDALLVPDKCKFLHQERMDMCESHLHWHTVAKESCGDRSMNLHDYGMLLPCGIDRFRGVEFVCCPADAGKESESAAVEEDDSDVWWGGAEADYTENSMTRDAAAEPAVLEDDEDADEEEDEDQDGDGDRDEKIEEEEEEEERTQSTSAALTSTTTTTTESVEEVVREVCFASAETGPCRAMLSRWYYVREERRCAPFIYGGCGGNRNNFESEEYCLSVCSGVLPTPSSSPPDAVDRYLETPADENEHAHFLKAKESLETKHRERMSQVMREWEEAERQAKSLPRNDKKAVIQHFQEKVEALEQESASERQQLVETHMARVEALLNDRRRLALESYLSALQADPPRPRHVFSLLKKYVRAEQKDRQHTLKHFEHVRMVDPKKAAQIRPQVLTHLRVIEERMNQSLGLLYKVPGVADDIQDQVELLQREQQEMSAQLANLQSDARVSYGNDALMPDSTAGLELLPAEDTQGFGFIHPESFNQPNTHNQVEPVDARPVPDLDLATRPVSGLKPDDIPELRMEAEERHSEVYHQKLVFFAEDVSSNKGAIIGLMVGGVVIATIIVITLVMLRKKQYTSIHHGIIEVDAAVTPEERHLSKMQQNGYENPTYKFFEQMHN

SEQ ID NO: 15 - protein, 887 aa, Drosophila melanogaster
  MISC_FEATURE: APPL
MCAALRRNLLLRSLWVVLAIGTAQVQAASPRWEPQIAVLCEAGQIYQPQYLSEEGRWVTDLSKKTTGPTCLRDKMDLLDYCKKAYPNRDITNIVESSHYQKIGGWCRQGALNAAKCKGSHRWIKPFRCLGPFQSDALLVPEGCLFDHIHNASRCWPFVRWNQTGAAACQERGMQMRSFAMLLPCGISVFSGVEFVCCPKHFKTDEIHVKKTDLPVMPAAQINSANDELVMNDEDDSNDSNYSKDANEDDLDDEDDLMGDDEEDDMVADEAATAGGSPNTGSSGDSNSGSLDDINAEYDSGEEGDNYEEDGAGSESEAEVEASWDQSGGAKVVSLKSDSSSPSSAPVAPAPEKAPVKSESVTSTPQLSASAAAFVAANSGNSGTGAGAPPSTAQPTSDPYFTHFDPHYEHQSYKVSQKRLEESHREKVTRVMKDWSDLEEKYQDMRLADPKAAQSFKQRMTARFQTSVQALEEEGNAEKHQLAAMHQQRVLAHINQRKREAMTCYTQALTEQPPNAHHVEKCLQKLLRALHKDRAHALAHYRHLLNSGGPGGLEAAASERPRTLERLIDIDRAVNQSMTMLKRYPELSAKIAQLMNDYILALRSKDDIPGSSLGMSEEAEAGILDKYRVEIERKVAEKERLRLAEKQRKEQRAAEREKLREEKLRLEAKKVDDMLKSQVAEQQSQPTQSSTQSQAQQQQQEKSLPGKELGPDAALVTAANPNLETTKSEKDLSDTEYGEATVSSTKVQTVLPTVDDDAVQRAVEDVAAAVAHQEAEPQVQHFMTHDLGHRESSFSLRREFAQHAHAAKEGRNVYFTLSFAGIALMAAVFVGVAVAKWRTSRSPHAQGFIEVDQNVTTHHPIVREEKIVPNMQINGYENPTYKYFEVKE

SEQ ID NO: 16 - protein, 680 aa, Caenorhabditis elegans
  MISC_FEATURE: APL1
MIGLLIPILVATVYAEGSPAGSKRHEKFIPMVAFSCGYRNQYMTEEGSWKTDDERYATCFSGKLDILKYCRKAYPSMNITNIVEYSHEVSISDWCREEGSPCKWTHSVRPYHCIDGEFHSEALQVPHDCQFSHVNSRDQCNDYQHWKDEAGKQCKTKKSKGNKDMIVRSFAVLEPCALDMFTGVEFVCCPNDQTNKTDVQKTKEDEDDDDDEDDAYEDDYSEESDEKDEEEPSSQDPYFKIANWTNEHDDFKKAEMRMDEKHRKKVDKVMKEWGDLETRYNEQKAKDPKGAEKFKSQMNARFQKTVSSLEEEHKRMRKEIEAVHEERVQAMLNEKKRDATHDYRQALATHVNKPNKHSVLQSLKAYIRAEEKDRMHTLNRYRHLLKADSKEAAAYKPTVIHRLRYIDLRINGTLAMLRDFPDLEKYVRPIAVTYWKDYRDEVSPDISVEDSELTPIIHDDEFSKNAKLDVKAPTTTAKPVKETDNAKVLPTEASDSEEEADEYYEDEDDEQVKKTPDMKKKVKVVDIKPKEIKVTIEEEKKAPKLVETSVQTDDEDDDEDSSSSTSSESDEDEDKNIKELRVDIEPIIDEPASFYRHDKLIQSPEVERSASSVFQPYVLASAMFITAICIIAFAITNARRRRAMRGFIEVDVYTPEERHVAGMQVNGYENPTYSFFDSKA

SEQ ID NO: 17 - protein, 650 aa, Homo sapiens
  MISC_FEATURE: APLP1
MGPASPAARGLSRRPGQPPLPLLLPLLLLLLRAQPAIGSLAGGSPGAAEAPGSAQVAGLCGRLTLHRDLRTGRWEPDPQRSRRCLRDPQRVLEYCRQMYPELQIARVEQATQAIPMERWCGGSRSGSCAHPHHQVVPFRCLPGEFVSEALLVPEGCRFLHQERMDQCESSTRRHQEAQEACSSQGLILHGSGMLLPCGSDRFRGVEYVCCPPPGTPDPSGTAVGDPSTRSWPPGSRVEGAEDEEEEESFPQPVDDYFVEPPQAEEEEETVPPPSSHTLAVVGKVTPTPRPTDGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSEHLGPSELEAPAPGGSSEDKGGLQPPDSKDDTPMTLPKGSTEQDAASPEKEKMNPLEQYERKVNASVPRGFPFHSSEIQRDELAPAGTGVSREAVSGLLIMGAGGGSLIVLSMLLLRRKKPYGAISHGVVEVDPMLTLEEQQLRELQRHGYENPTYRFLEERP

SEQ ID NO: 18 - protein, 763 aa, Homo sapiens
  MISC_FEATURE: APLP2
MAATGTAAAAATGRLLLLLLVGLTAPALALAGYIEALAANAGTGFAVAEPQIAMFCGKLNMHVNIQTGKWEPDPTGTKSCFETKEEVLQYCQEMYPELQITNVMEANQRVSIDNWCRRDKKQCKSRFVTPFKCLVGEFVSDVLLVPEKCQFFHKERMEVCENHQHWHTVVKEACLTQGMTLYSYGMLLPCGVDQFHGTEYVCCPQTKIIGSVSKEEEEEDEEEEEEEDEEEDYDVYKSEFPTEADLEDFTEAAVDEDDEDEEEGEEVVEDRDYYYDTFKGDDYNEENPTEPGSDGTMSDKEITHDVKAVCSQEAMTGPCRAVMPRWYFDLSKGKCVRFIYGGCGGNRNNFESEDYCMAVCKAMIPPTPLPTNDVDVYFETSADDNEHARFQKAKEQLEIRHRNRMDRVKKEWEEAELQAKNLPKAERQTLIQHFQAMVKALEKEAASEKQQLVETHLARVEAMLNDRRRMALENYLAALQSDPPRPHRILQALRRYVRAENKDRLHTIRHYQHVLAVDPEKAAQMKSQVMTHLHVIEERRNQSLSLLYKVPYVAQEIQEEIDELLQEQRADMDQFTASISETPVDVRVSSEESEEIPPFHPFHPFPALPENEDTQPELYHPMKKGSGVGEQDGGLIGAEEKVINSKNKVDENMVIDETLDVKEMIFNAERVGGLEEERESVGPLREDFSLSSSALIGLLVIAVAIATVIVISLVMLRKRQYGTISHGIVEVDPMLTPEERHLNKMQNHGYENPTYKYLEQMQI